(19) United States
(12) Patent Application Publication
(10) Pub. No.: US 2011/0288103 A1
(43) Pub. Date: Nov. 24, 2011

(54) COMPOUNDS, COMPOSITIONS, AND METHODS COMPRISING 1,3,4-OXADIAZOLE DERIVATIVES

(75) Inventors: Eugenio L. de Hostos, South San Francisco, CA (US); Tue H. Nguyen, South San Francisco, CA (US)

(73) Assignee: Institute for OneWorld Health

(21) Appl. No.: 13/090,115

(22) Filed: Apr. 19, 2011

Related U.S. Application Data
(60) Provisional application No. 61/326,173, filed on Apr. 20, 2010.

(51) Int. Cl. A61K 31/496; A61K 31/4245; A61K 31/454; A61P 11/00; A61P 25/00; A61P 31/00; A61P 11/06; A61P 1/00; A61P 1/12; A61P 9/06; A61P 15/08; A61P 9/10; A61P 13/02; A61P 3/00; A61P 13/12; A61P 9/12; C12N 5/07

(52) U.S. Cl. 514/254.03; 435/375; 514/364; 514/326

(57) ABSTRACT

The present invention relates to methods for treating a disease in an animal, which disease is responsive to blocking of calcium activated chloride echannel (CaCC) by administering to a mammal in need thereof an effective amount of a compound defined herein (including those compounds set forth in Tables 1-2 or encompassed by formulas I-IV) or compositions thereof, thereby treating the disease.

### CROSS REFERENCE TO RELATED APPLICATION

This application claims priority under 35 U.S.C. 119(e) to U.S. provisional patent application No. 61/326,173 filed Apr. 20, 2010, which is incorporated herein in its entirety by reference.

### FIELD OF THE INVENTION

This application and invention discloses 1,3,4-oxadiazole-containing compounds that inhibit the transport of ions (e.g., chloride ions) across cell membranes containing calcium activated chloride channels. The structures of the compounds and derivatives thereof, as well as pharmaceutical formulations and methods of use are described in more detail below.

### BACKGROUND

Ion channels are not only essential for normal cellular functions but also play a critical role in numerous diseased states. For example, cystic fibrosis results when ion transport in epithelial cells of individuals is altered due to a genetic defect of the cystic fibrosis transmembrane conductance regulator (CFTR; Knowles et al., 1983, J. Clin. Invest. 71:1410-1417). Although serious airway pathology may usually be the primary cause of mortality in young adults with CF, intestinal epithelial alterations may also be observed. The severity of tissue lesions may not correlate exclusively with the expression of CFTR in humans or mice, suggesting the involvement of cell-specific channels in addition to CFTR. Further support for the involvement of other channel protein molecules in CF comes from observations that airway CaCCs are found to be up-regulated in cystic fibrosis, providing an alternative chloride conductance to compensate for missing or defective CFTR.

Calcium-activated chloride channels (CaCCs) are an addition to the family of chloride conductance proteins. CaCCs possess multifunctional capability and have been shown not only to be anion channels but also to mediate cell adhesion (Abdel-Ghany et al. 2001 J. Biol Chem 276:25438-25446). In particular, the human isoform, hCLCA2, when expressed in the lung, is believed to play a role in modulating the severity of cystic fibrosis (Gruber et al. 1999 Am J Physiol Cell Physiol 276:C1261-1270). It is also a key molecule in the adhesion of tumor cells to lung endothelia (Abdel-Ghany et al. 2001, supra) and in the tumorigenicity of human breast cancer (Gruber and Pauli, 1999 Cancer Res 59:5488-5491).

CaCCs regulate sensory transduction, epithelial secretion, neuronal excitability, smooth muscle contraction and vascular tone (Hartzell et al. 2005 Annu. Rev. Physiol. 67:719-58). CaCCs have been implicated in a wide range of important physiological functions including the high-gain, low-noise amplification in olfactory transduction, taste adaptation, control of action potential waveform in neurons, membrane potential stabilization in photoreceptors, modulation of fluid secretion from glands and airway epithelia, and positive feedback regulation of smooth muscle contraction induced by G protein-coupled receptors (GPCRs). Notwithstanding the multitude of CaCC types reported, CaCCs are found in many different cell types including Xenopus oocytes, secretory epithelial cells, hepatocytes, pulmonary artery endothelial cells, and vascular, airway and gut smooth muscles.

Despite the fact that CaCCs are so broadly expressed in cells and play such important functions, understanding these channels has been limited by the absence of specific blockers. Thus, a need exists for methods that block the ion transport through the chloride channels and are useful for the treatment of diseases responsive to such blockage.

### SUMMARY OF THE INVENTION

This invention is directed to one or more of compounds, compositions and methods which are useful in treating diseases that are responsive to the blocking of a calcium activated chloride channel (CaCC).

In one aspect, this invention provides a method of treating a disease in an animal, which disease is responsive to blocking of a calcium activated chloride channel in the animal, comprising or alternatively consisting essentially of, or alternatively consisting of administering to an animal in need thereof an effective amount of a compound of formula I:

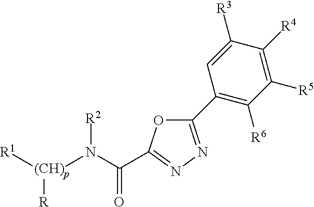

wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
or R¹ and R² are taken together with the nitrogen atom to which hey are bonded to form a heterocycle or substituted heterocycle;
R³, R⁴, R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;

or a pharmaceutically acceptable salt, isomer, or tautomer thereof,

thereby treating the disease in the animal.

In another aspect, this invention provides a method for blocking a transport of a halide ion across a calcium activated chloride channel (CaCC), comprising or alternatively consisting essentially of or alternatively consisting of, contacting the CaCC with an effective amount of a compound of formula I:

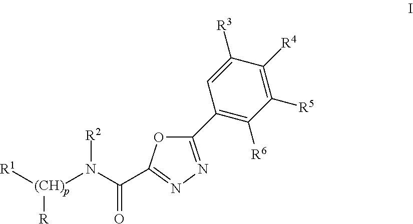

wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² are taken together with the nitrogen atom to which they are bonded to form a heterocycle or substituted heterocycle;
R³, R⁴, and R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;

or a pharmaceutically acceptable salt, isomer, or tautomer thereof,

thereby blocking the transport of the halide ion across the CaCC.

In another aspect, this invention provides an in vitro method for blocking a transport of a halide ion across a calcium activated chloride channel (CaCC), comprising or alternatively consisting essentially of, or alternatively consisting of, contacting the CaCC with an effective amount of a compound of formula I:

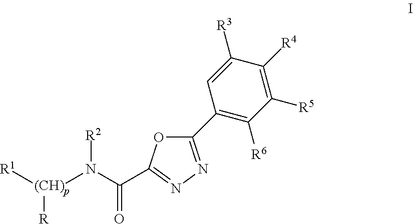

wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² are taken together with the nitrogen atom to which they are bonded to form a heterocycle or substituted heterocycle;
R³, R⁴, and R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;

or a pharmaceutically acceptable salt, isomer, or tautomer thereof,

thereby blocking the transport of the halide ion across the CaCC.

Specific aspects of the methods described above comprise use of one or more of the compounds set forth in Tables 1-2 or encompassed by formulas I-IV or compositions comprising these compounds.

### BRIEF DESCRIPTION OF THE FIGURES

FIG. 1 depicts a blocking of CaCC-mediated Cl⁻ currents by addition of compound 10 where intracellular calcium was stably buffered.

FIG. 2 depicts a blocking of CaCC-mediated Cl⁻ currents by addition of compound 10 where intracellular calcium was unbuffered.

### DETAILED DESCRIPTION OF THE INVENTION

The invention provides methods of using 1,3,4-oxadiazole-containing compounds to inhibit or block one or more of CaCC. The compounds and derivatives thereof, as well as compositions, pharmaceutical formulations and methods of use, are further provided by the invention.

Throughout this application, the various embodiments are only exemplary and should not be construed as descriptions of alternative species. Rather it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

### A. Definitions

The practice of the present invention will employ, unless otherwise indicated, conventional techniques of organic chemistry, pharmacology, immunology, molecular biology, microbiology, cell biology and recombinant DNA, which are within the skill of the art. See, e.g., Sambrook. Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Flames and C. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

“Animal” of diagnosis or treatment refers to an animal such as a mammal, or a human, ovine, bovine, feline etc. Non-human animals subject to diagnosis or treatment include, for example, simians, murine, such as, rat, mice, canine, leporid, livestock, sport animals, and pets.

The term “blocking” refers to a decrease or an inhibition of the activity of the CaCC by at least about 10%, or alternatively at least about 20%, or alternatively at least about 25%, or alternatively at least about 30%, or alternatively at least about 35%, or alternatively at least about 40%, or alternatively at least about 45%, or alternatively at least about 50%, or alternatively at least about 55%, or alternatively at least about 60%, or alternatively at least about 65%, or alternatively at least about 70%, or alternatively at least about 80%, or alternatively at least about 90%, or alternatively at least about 99%, or alternatively at least about 100%, compared to the activity of CaCC in the absence of the compounds, described herein.

The term “calcium activated chloride channel” refers to the chloride channel whose conductance is activated by calcium. In some embodiments, for the in vitro methods provided herein the chloride channel is activated with calcium prior to contact with the compound.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding, more than trace elements of other ingredients. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such are known in the art.

The terms “polypeptide” and “protein” are synonymously used in their broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency.” In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region for a polypeptide or polypeptide region) has a certain percentage (for example, 80%, 85%, 90%, or 95%) of “sequence identity” to another sequence when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+MR. Details of these programs can be found at the following Internet address: http://www.ncbi.nlm.nih.gov/cgi-bin/BLAST.

A variety of sequence alignment software programs are available in the art. Non-limiting examples of these programs are BLAST family programs including BLASTN, BLASTP, BLASTX, TBLASTN, and TBLASTX (BLAST is available from the worldwide web at ncbi.nlm.nih.gov/BLAST/), FastA, Compare, DotPlot, BestFit, GAP, FrameAlign, ClustalW, and Pileup. These programs are obtained commercially available in a comprehensive package of sequence analysis software such as GCG Inc.'s Wisconsin Package. Other similar analysis and alignment programs can be purchased from various providers such as DNA Star's MegAlign, or the alignment programs in GeneJockey. Alternatively, sequence analysis and alignment programs can be accessed through the world wide web at sites such as the CMS Molecular Biology Resource at sdsc.edu/ResTools/cmshp.html. Any sequence database that contains DNA or protein sequences corresponding to a gene or a segment thereof can be used for sequence analysis. Commonly employed databases include but are not limited to GenBank, EMBL, DDRJ, PDB, SWISS-PROT, EST, STS, GSS, and HTGS.

Parameters for determining the extent of homology set forth by one or more of the aforementioned alignment programs are known. They include but are not limited to p value, percent sequence identity and the percent sequence similarity. P value is the probability that the alignment is produced by chance. For a single alignment, the p value can be calculated according to Karlin et al. (1990) PNAS 87:2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl(CH₃—), ethyl(CH₃CH₂—), n-propyl (CH₃CH₂CH₂—), isopropyl((CH₃)₂CH—), n-butyl(CH₃CH₂CH₂CH₂—), isobutyl((CH₃)₂CHCH₂—), sec-butyl((CH₃)(CH₃CH₂)CH—), t-butyl((CH₃)₃C—), n-pentyl(CH₃CH₂CH₂CH₂CH₂—), and neopentyl((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein. For example, in some embodiments, substituted alkyl is substituted with fluoro such as trifluomethyl, or substituted with substituted aryl.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxyl, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy,

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted, heteroaryl-C(O)—, heterocyclic-C(O)—, and substituted heterocyclic-C(O)—, wherein alkyl, substituted alkyl, alkenyl, substituted alkenyl, ally substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁷C(O)alkyl, —NR⁴⁷C(O) substituted alkyl, —NR⁴⁷C(O)cycloalkyl, —NR⁴⁷C(O) substituted cycloalkyl, —NR⁴⁷C(O)cycloalkenyl, —NR⁴⁷C(O) substituted cycloalkenyl, —NR⁴⁷C(O)alkenyl, —NR⁴⁷C(O) substituted alkenyl, —NR⁴⁷C(O)alkynyl, —NR⁴⁷C(O) substituted alkynyl, —NR⁴⁷C(O)aryl, —NR⁴⁷C(O) substituted aryl, —NR⁴⁷C(O)heteroaryl, —NR⁴⁷C(O) substituted heteroaryl, —NR⁴⁷C(O)heterocyclic, and —NR⁴⁷C(O) substituted heterocyclic wherein R⁴⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O, substituted heteroaryl-C(O)O—, heterocyclic-C(O)O—, and substituted heterocylic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR⁴⁸R⁴⁹ where R⁴⁸ and R⁴⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl-SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁸ and R⁴⁹ are optionally joined, together with the nitrogen bound thereto to form a heterocyclic or substituted heterocyclic group, provided that R⁴⁸ and R⁴⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴⁸ is hydrogen and R⁴⁹ is alkyl, the substituted amino group is sometimes referred to herein as alkylamino. When R⁴⁸ and R⁴⁹ are alkyl, the substituted amino group is sometimes referred to herein as dialkylamino. When referring to a monosubstituted amino, it is meant that either R⁴⁸ or R⁴⁹ is hydrogen but not both. When referring to a disubstituted amino, it is meant that neither R⁴⁸ nor R⁴⁹ are hydrogen.

“Aminocarbonyl” refers to the group —C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁴⁷C(O)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR⁴⁷C(S)NR⁵⁰R⁵¹ where R is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein,

“Aminosulfonyloxy” refers to the group —O—SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR⁴⁷SO₂NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁵²)NR⁵⁰R⁵¹ where R⁵⁰, R⁵¹, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to an groups which are substituted with 1 to 5, preferably 1 to 3, or more preferably 1 to 2 substituents selected from the group consisting of alkyl, substituted alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbony (amino, aminothiocarbonylamino, aminocarbonyloxy, aminosulfonyl, aminosulfonyloxy, aminosulfonylamino, amidino, aryl, substituted aryl, aryloxy, substituted aryloxy, air substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein,

“Substituted arylthio” refers to the group —S-(substituted aryl), where substituted aryl is as defined herein.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR⁴⁷C(O)O-alkyl, —NR⁴⁷C(O)O-substituted alkyl, —NR⁴⁷C(O)O-alkenyl, —NR⁴⁷C(O)O-substituted alkenyl, —NR⁴⁷C(O)O-alkynyl, —NR⁴⁷C(O)O-substituted alkynyl, —NR⁴⁷C(O)O-aryl, —NR⁴⁷C(O)O-substituted aryl, —NR⁴⁷C(O)O-cycloalkyl, —NR⁴⁷C(O)O-substituted cycloalkyl, —NR⁴⁷C(O)O-cycloalkenyl, —NR⁴⁷C(O)O-substituted cycloalkenyl, —NR⁴⁷C(O)O-heteroaryl, —NR⁴⁷C(O)O-substituted heteroaryl, —NR⁴⁷C(O)O-heterocyclic, and —NR⁴⁷C(O)O-substituted heterocyclic wherein R⁴⁷ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Cycloalkyloxy” refers to —O-cycloalkyl.

“Substituted cycloalkyloxy refers to —O—(substituted cycloalkyl).

“Cycloalkylthio” refers to —S-cycloalkyl.

“Substituted cycloalkylthio” refers to —S-(substituted cycloalkyl),

“Cycloalkenyloxy” refers to —O-cycloalkenyl.

“Substituted cycloalkenyloxy” refers to —O— (substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁵³C(═NR⁵³)N(R⁵)₂ where each R⁵³ is independently selected from the group consisting of hydrogen, alkyl, substituted alkyl, aryl, substituted aryl, heteroaryl, substituted heteroaryl, cycloalkyl, substituted cycloalkyl, heterocyclic, and substituted heterocyclic and two R⁵³ groups attached to a common guanidino nitrogen atom are optionally joined together with the nitrogen bound thereto to form a heterocyclic or substituted heterocyclic group, provided that at least one R⁵³ is not hydrogen, and wherein said substituents are as defined herein.

“Halo” or “halogen” refers to fluoro, chloro, bronco and iodo.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through a non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, oxadiazole, pyridine, pyrazine, pyrimidine, isoxazole, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spirocycloalkyl” and “Spiro ring systems” refers to divalent cyclic groups from 3 to 10 carbon atoms having a cycloalkyl or heterocycloalkyl ring with a Spiro union (the union formed by a single atom which is the only common member of the rings) as exemplified by the following structure:

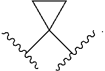

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—.

“Substituted sulfonyloxy” refers to the group —OSO₂-alkyl, —OSO₂-substituted alkyl, —OSO₂-alkenyl, —OSO₂-substituted alkenyl, —OSO₂-substituted cylcoalkyl, —OSO₂-cycloalkenyl, —OSO₂-substituted cycloalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfonylamino” refers to the group —NR⁵⁰SO₂R⁵¹, wherein R⁵⁰ and R⁵¹ independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thioxo” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Isomer” refers to tautomerism, conformational isomerism, geometric isomerism, stereoisomerism and/or optical isomerism. For example, the compounds and prodrugs of the invention may include one or more chiral centers and/or double bonds and as a consequence may exist as stereoisomers, such as double-bond isomers (i.e., geometric isomers), enantiomers, diasteromers, and mixtures thereof, such as racemic mixtures. As another example, the compounds and prodrugs of the invention may exist in several tautomeric forms, including the enol form, the keto form, and mixtures thereof.

“Stereoisomer” or “stereoisomers” refer to compounds that differ in the china or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as oxadiazoles, imidazoles, benzimidazoles, triazoles, and tetrazoles.

“Prodrug” refers to art recognized modifications to one or more functional groups which functional groups are metabolized in vivo to provide a compound of this invention or an active metabolite thereof. Such functional groups are well known in the art including acyl or thioacyl groups for hydroxyl and/or amino substitution, conversion of one or more hydroxyl groups to the mono-, di- and tri-phosphate wherein optionally one or more of the pendent hydroxyl groups of the mono-, di- and tri-phosphate have been converted to an alkoxy, a substituted alkoxy, an aryloxy or a substituted aryloxy group, and the like.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate (see Stahl and Wermuth, eds., “HANDBOOK OF PHARMACEUTICALLY ACCEPTABLE SALTS,” (2002), Verlag Helvetica Chimica Acta, Zürich, Switzerland), for an extensive discussion of pharmaceutical salts, their selection, preparation, and use.

Generally, pharmaceutically acceptable salts are those salts that retain substantially one or more of the desired pharmacological activities of the parent compound and Which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion, or an aluminum ion) or coordinates with an organic base (e.g., ethanolamine, diethanolamine, triethanolamine. N-methylglucamine, morpholine, piperidine, dimethylamine, diethylamine, triethylamine, and ammonia).

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers or other compounds arrived at by defining substituents with further substituents to themselves (e.g., substituted aryl having a substituted and group or another group as a substituent which is itself substituted with a substituted aryl group or another group, which is further substituted by a substituted aryl group or another group etc.) are not intended for inclusion herein. In such cases, the maximum number of such substitutions is four. For example, serial substitutions of substituted aryl groups with two other substituted aryl groups are limited to -substituted aryl-(substituted aryl)-substituted aryl-(substituted aryl).

Similarly, it is understood that the above definitions are not intended to include impermissible substitution patterns (e.g., methyl substituted with 5 fluoro groups). Such impermissible substitution patterns are well known to the skilled artisan.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases such as, but not limited to, cardiac disease, diarrhea or PKD. In general, one will desire to administer an amount of the compound that is effective to achieve a serum level commensurate with the concentrations found to be effective in vitro. Thus, where a compound is found to demonstrate in vitro activity, for example as noted in the Tables discussed below one can extrapolate to an effective dosage for administration in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response such as inhibiting or blocking CaCC.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Preferred are compounds that are potent and can be administered locally at very low doses, thus minimizing systemic adverse effects.

### B. Methods of the Invention

In one aspect, there is provided a method of treating a disease in an animal, which disease is responsive to blocking of a calcium activated chloride channel (CaCC) in the animal, by administering to the animal in need thereof an effective amount of a compound, as described herein. In another aspect, there is provided a method for blocking a transport of a halide ion across a calcium activated chloride channel, by contacting the CaCC with an effective amount of a compound, as described herein. In one aspect, the channel has been activated prior to contacting the channel with the compound provided herein. The channel may be activated by several factors including, but are not limited to, voltage, Ca²⁺, extracellular ligands, and pH. In some embodiments, the methods of the invention are practiced in vitro, in vivo, or ex vivo. The compounds disclosed herein are useful in the treatment of a condition, disorder or disease or symptom of such condition, disorder, or disease, where the condition, disorder or disease is responsive to blocking of CaCC. In one aspect, the methods of the invention treat the diseases by inhibiting or blocking ion transport, e.g. HCO₃⁻ or halide ion, e.g., chloride ion, transport by CaCC. In some embodiments, the channels are present in animal cell membranes. In some embodiments, the channels are present in mammalian cell membranes. In some embodiments, the animal cell or the mammalian cell includes, but is not limited to, epithelial cell, bipolar cell, smooth muscle cell, acinar and duct cell of lachrymal, parotid, submandibular, and/or sublingual gland, endothelial cell, or kidney cell.

Calcium-activated chloride channels (CaCCs) play important role in cellular physiology, including epithelial secretion of electrolytes and water, sensory transduction, regulation of neuronal and cardiac excitability, and regulation of vascular tone. See Hartzell et al. supra.; Kotlikoff and Wang, Am J Respir Crit Care Med 158:S109-S114 (1998); and Connon et al. J of Histochem. And Cytochem. 52 (3):415-418 (2004).

Examples of the diseases that are responsive to blocking of the CaCC, as in the methods of the present invention, are as described below.

Vertebrate olfactory receptor neurons express CaCCs that play a role in transduction of olfactory stimuli. Odorants may bind to and activate G protein—coupled receptors in the ciliary membrane of olfactory receptor neurons. These receptors may activate adenylyl cyclase, which may produce cAMP and turn on cyclic-nucleotide-gated channels that are permeable to both Na⁺ and Ca²⁺. This may lead to a membrane depolarization and an elevation of [Ca²⁺]ᵢ in the cilium, which may activate CaCCs. The Cl⁻ efflux (inward current) may depolarize the membrane further. Thus, in olfactory receptor neurons, the Cl⁻ efflux through CaCCs may serve as an amplification system of the odorant-activated current. The physiological role of the amplification could serve to increase the signal-to-noise ratio and hence to increase sensitivity to odorants. Further, CaCCs are present in both mammalian and amphibian taste receptors. Taste stimuli produce a depolarizing current in taste receptor cells that may result in a discharge of action potentials. The action potentials in the taste receptors are followed by an outward current that is mediated by CaCCs, which open in response to Ca²⁺ influx during the action potentials. Therefore, CaCC's play a role in olfactory and taste disorders.

In some embodiments, the methods of the invention are used to treat, prevent or alleviate olfactory and taste disorders that are responsive to blocking of CaCC or its activity. The olfactory diseases include, but are not limited to, smell and taste disorder such as, anosmia—inability to detect odors; hyposmia decreased ability to detect odors; dysosmia—distorted identification of smell; parosmia—altered perception of smell in the presence of an odor, usually unpleasant; phantosmia perception of smell without an odor present; agnosia—inability to classify or contrast odors, although able to detect odors; ageusia—inability to taste; hypogeusia—decreased ability to taste; and dysgeusia—distorted ability to taste.

In some embodiments, the methods of the invention are used to treat stroke. The stroke includes stroke caused by ischemia. Increased activation of excitatory amino acid (EAA) receptors may be a cause of neuronal damage in ischemia and large increases in EAA concentrations in the extracellular space may occur during ischemia. The compounds provided herein that block the chloride channel may lead to reduced EAA release in vitro and in vivo.

The inner segments of rods and cones in the retina may express CaCCs. In addition, CaCCs may also be present in the synaptic terminal of bipolar cells. In some embodiments, the methods of the invention are used to treat, prevent or alleviate an ophthalmic angiogenesis related disease, such as, but are not limited to, exudative macular degeneration, age-related macular degeneration (AMD), retinopathy, diabetic retinopathy, proliferative diabetic retinopathy, diabetic macular edema (DME), ischemic retinopathy (e.g. retinal vain or artery occlusion), retinopathy of prematurity, neovascular glaucoma, and corneal neovascularization.

CaCCs are expressed in a variety of different neurons, including dorsal root ganglion (DRG) neurons, spinal cord neurons, and autonomic neurons. About 45-90% of the somatosensory neurons from the DRG that sense skin temperature, touch, muscle tension, and pain, may express CaCCs. In some embodiments, the methods of the invention are used to treat, prevent or alleviate neuronal disorders that are responsive to blocking of CaCC or its activity. The neuronal disorders include, but are not limited to, myotonia congenital, myotonia dystrophy, epilepsy, cerebrovascular accident (stroke), Parkinson's disease, multiple sclerosis, myasthenia gravis, Huntington's disease (Huntington's chorea), Creutzfeldt-Jakob disease, amyotrophic lateral sclerosis, black widow spider, blepharospasm, complex repetitive discharges, Crisponi syndrome, dystonia variants, fasciculations, geniospasm, hemifacial spasm, Isaac's Syndrome, motor neuron disorders, motor neuropathies, myokymia, neuromyotonia, palmaris brevis spasm, polyneuropathy, vascular disease of spinal chord, startle syndrome (hyperekplexia), strychnine, Stiffman Syndrome, superior oblique myokymia, tetanus, tetany, tremor, and Whipple's.

CaCCs also play a role in repolarization of the cardiac action potential. In some embodiments, the compounds of the invention are used to treat, prevent or alleviate a cardiovascular disease, such as, but not limited to, atherosclerosis, ischemia, reperfusion injury, hypertension, restenosis, arterial inflammation, myocardial ischaemia and ischaemic heart disease.

CaCC has been implicated in the pathophysiology of asthma. In some embodiments, the methods of the invention are used to treat, prevent or alleviate asthma.

Airway epithelia use ion transport mechanisms to control the level of airway surface liquid, which may be important for mucous hydration and protection against infection. Secretion of fluid into the airway is accomplished by basally located transporters that accumulate Cl⁻ in the cell against the Cl⁻ electrochemical gradient and by apical Cl⁻ channels that permit Cl⁻ to flow into the extracellular space down its electrochemical gradient. Airway epithelial cells as well as intestinal epithelia express CaCCs in their membrane.

In some embodiments, the methods of the invention are used to treat, prevent or alleviate an obstructive or inflammatory airway disease, such as airway hyperreactivity, pneumoconiosis, aluminosis, anthracosis, asbestosis, chalicosis, ptilosis, siderosis, silicosis, tabacosis, byssinosis, sarcoidosis, berylliosis, pulmonary emphysema, acute respiratory distress syndrome (ARDS), acute lung injury (ALI), acute or chronic infectious pulmonary disease, chronic obstructive pulmonary disease (COPD), bronchitis, chronic bronchitis, wheezy bronchitis, excerbation of airways hyperreactivity or cystic fibrosis, or cough including chronic cough, excerbation of airways hyperreactivity, pulmonary fibrosis, pulmonary hypertension, inflammatory lung diseases, and acute or chronic respiratory infectious diseases.

In some embodiments, the methods of the invention are used to treat, prevent or alleviate diarrhea and/or urinary incontinence.

As used herein, “diarrhea” intends a medical syndrome which is characterized by the primary symptom of diarrhea (or scours in animals) and secondary clinical symptoms that may result from a secretory imbalance and without regard to the underlying cause and therefore includes exudative (inflammatory), decreased absorption (osmotic, anatomic derangement, and motility disorders) and secretory. All forms of diarrhea have a secretory component. Symptoms include, but are not limited to impaired colonic absorption, ulcerative colitis, shigellosis, and amebiasis. Osmotic diarrhea can occur as a result of digestive abnormalities such as lactose intolerance Anatomic derangement results in a decreased absorption surface caused by such procedures as subtotal colectomy and gastrocolic fistula. Motility disorders result from decreased contact time resulting from such diseases as hyperthyroidism and irritable bowel syndrome. Secretory diarrhea is characterized by the hypersecretion of fluid and electrolytes from the cells of the intestinal wall. In classical form, the hypersecretion is due to changes which are independent of the permeability, absorptive capacity and exogenously generated osmotic gradients within the intestine. However, all forms of diarrhea can manifest a secretory component.

Diarrhea may be caused by infection by a variety of bacteria, parasites and viruses and may be a threat to regions lacking potable water. Preventing exposure to the pathogens responsible for diarrhea may be the only way to avert infection. This may require massive improvement in both sanitation and nutritional status in developing countries, which may be unlikely to occur in the short term. Thus, it is a continuing threat to the third world and especially the health of children who may lack a robust immune response. Many who do survive may have lasting health problems due to the effects of recurrent infections and malnutrition. Diarrheal diseases may also be the major cause of childhood hospitalization, primarily for dehydration.

Diarrhea amenable to treatment using the compounds of the invention can result from exposure to a variety of pathogens or agents including, without limitation, cholera toxin (Vibrio cholera), E. coli (particularly enterotoxigenic (ETEC)), Salmonella, e.g. Cryptosporidiosis, diarrheal viruses (e.g., rotavirus)), food poisoning, or toxin exposure that results in increased intestinal secretion mediated by CaCC.

Other diarrheas that can be treated by the methods of the invention include diarrhea associated with AIDS (e.g., AIDS-related diarrhea), diarrheas caused by anti-AIDS medications such as protease inhibitors and inflammatory gastrointestinal disorders, such as ulcerative colitis, inflammatory bowel disease (IBD). Crohn's disease, chemotherapy, and the like. It has been reported that intestinal inflammation modulates the expression of three major mediators of intestinal salt transport and may contribute to diarrhea in ulcerative colitis both by increasing transepithelial Cl⁻ secretion and by inhibiting the epithelial NaCl absorption. See, e.g., Lohi et al. (2002) Am. J. Physiol. Gastrointest. Liver Physiol 283 (3):G567-75.

Diarrheal episodes can be either acute or persistent (lasting two weeks or more). Diarrheal diseases may have other effects, such as reduced growth, reduced appetite, altered feeding patterns, decreased absorption of nutrients, reduced fitness, reduced cognitive function, and reduced school performance. The primary cause of death from diarrhea may be dehydration. As dehydration worsens, symptoms may progress from thirst, restlessness, decreased skin turgor and sunken eyes to diminished consciousness, rapid and feeble pulse and low or undetectable blood pressure. Diarrhea also may arise as a result of coinfection with other diseases such as malaria, and HIV and may be a comorbidity factor associated with deaths due to these diseases.

In some embodiments, the methods of the invention are used to treat, prevent or alleviate a kidney disease. Examples of kidney diseases include, but are not limited to, renal tubular disorders such as, but are not limited to, hypercalciuric nephrolithiasis, x-linked recessive nephrolithiasis, dent disease; nephrogenic diabetes insipidus; and Bartter syndrome (hypokalemic alkalosis with hypercalciuria).

The methods of the invention can also treat polycystic kidney disease (PKD) and associated diseases or disorders such as autosomal dominant polycystic kidney disease (ADPKD), autosomal recessive polycystic kidney disease and acquired cystic kidney disease. The manifestation of PKD may be the progressive cystic dilation of renal tubules which ultimately may lead to renal failure in half of affected individuals. PKD-associated renal cysts may enlarge to contain several liters of fluid and the kidneys may enlarge progressively causing pain. Other abnormalities such as hematuria, renal and urinary infection, renal tumors, salt and water imbalance and hypertension may frequently result from the renal defect. Cystic abnormalities in other organs, including the liver, pancreas, spleen and ovaries may be found in PKD. Massive liver enlargement may cause portal hypertension and hepatic failure.

In some embodiments, the methods of the invention are used to treat, prevent or alleviate a bone metabolic disease, such as an osteoclast related bone disease, such as osteoporosis, postmenopausal osteoporosis, secondary osteoporosis, osteolytic breast cancer bone metastasis, osteolytic cancer invation, or Paget's disease of hone.

In some embodiments, the methods of the invention are used to treat, prevent or alleviate diseases that are responsive to inhibition of angiogenesis, such as diseases that involve the proliferation of tumor cells, such as, but are not limited to, cancer, metastatic cancer, prostate cancer, lung cancer, breast cancer, bladder cancer, renal cancer, colon cancer, gastric cancer, pancreatic cancer, ovarian cancer, melanoma, hepatoma, sarcoma, and lymphoma.

In some embodiments, the methods of the invention are used to treat, prevent or alleviate disease, disorder or condition that is responsive to reduction of intraocular pressure, such as ocular hypertension, open-angle glaucoma, chronic open-angle glaucoma, angle-closure glaucoma and ciliary injection caused by angle-closure glaucoma, rheumatoid arthritis, and sickle-cell anaemia.

In some embodiments of the methods of the invention, CaCC is CLCA1, CLCA2, or CLCA4 or homologs thereof. The calcium-activated chloride channel CLCA1, the calcium-activated chloride channel CLCA2, the calcium-activated chloride channel CLCA4, and lung-endothelial cell adhesion molecule-1 (Lu-ECAM-1) are members of a family of proteins that appear to mediate a calcium-activated chloride conductance in a variety of tissues. These proteins may share high degrees of homology in size, sequence (75 to 89% identity), and predicted structure, but may differ significantly in their tissue distributions. In some embodiments, the calcium activated chloride channel is human CLCA1 and/or CLCA2 and/or CLCA4.

CLCA1 is a protein that in humans is encoded by the CLCA1 gene. All members of this gene family may map to the same region on chromosome 1p31-p22 and may share a high degree of homology in size, sequence, and predicted structure, but may differ significantly in their tissue distributions. The encoded protein may be expressed as a precursor protein that may be processed into two cell-surface-associated subunits. The encoded protein may be involved in mediating calcium-activated chloride conductance in the intestine.

CLCA2 is a protein that in humans is encoded by the CLCA2 gene. All members of this gene family may also map to the same site on chromosome 1p31-p22 and may share high degree of homology in size, sequence and predicted structure, but may differ significantly in their tissue distributions. Since this protein is expressed predominantly in trachea and lung, it may play a role in the complex pathogenesis of cystic fibrosis. It may serve as adhesion molecule for lung metastatic cancer cells, mediating vascular arrest and colonization, and may also act as a tumor suppressor gene for breast cancer. For example, target CLCA2 proteins are hCLCA2 and homologs thereof, particularly functional homologs or fragment thereof, e.g., mCLCA4, etc. By functional homolog thereof is meant that the homolog has substantially the same mucin secretion modulatory activity, particularly respiratory system cell mucin secretion modulatory activity, as hCLCA2.

In many embodiments, the subject homologs are proteins whose amino acid sequence is at least about 55%, usually at least about 75% and more usually at least about 90% identical and/or at least about 60% similar, usually at least about 75% and more usually at least about 90% similar over at least a substantial portion of its length, e.g., at least about 50%, usually at least about 75% and more usually at least about 90%, and often at least about 95% and higher, with the amino acid sequence of hCLCA2, and in many embodiments with the sequence of hCLCA2 as reported in Genbank Accession Nos. AX054697, AF043977, AB026833, AF127980 and Z24653.

In one aspect, the compounds and compositions in the methods of the invention are administered or delivered to treat the diseases as provided herein and/or associated symptoms in an animal in need of such treatment. The term “animal” is used broadly to include mammals such as a human patient or other farm animals in need of such treatment. In one aspect, the animal is an infant (i.e., less than 2 years old, or alternatively, less than one year old, or alternatively, less than 6 months old, or alternatively, less than 3 months old, or alternatively, less than 2 months old, or alternatively, less than 1 one month old, or alternatively, less than 2 weeks old), a newborn (e.g., less than one week old, or alternatively, less than one day old), a pediatric patient (e.g., less than 18 years old or alternatively less than 16 years old) or yet further, a geriatric patient (e.g., greater than 65 years old).

In one aspect, the methods of the invention are used in the treatment of the conditions as described above by administering art effective amount of the compound defined herein (including those compounds set forth in Tables 1-2 or encompassed by formulas I-IV) or compositions thereof.

In one embodiment, this invention provides use of a compound of formula I, II, III, or IV or compounds of Tables 1-2 or a composition comprising a compound of formula I, II, III, or IV or compounds of Tables 1-2 for treating a disease in art animal, which disease is responsive to blocking of a calcium activated chloride channel (CaCC) in the animal, comprising administering to an animal in need thereof an effective amount of a compound of formula I, II, III, or IV or compounds of Tables 1-2 or a composition comprising a compound of formula I, II, III, or IV or compounds of Tables 1-2, thereby treating the disease.

In another embodiment, this invention provides use of a compound of formula I, II, III, or IV or compounds of Tables 1-2 or a composition comprising a compound of formula I, II, III, or IV or compounds of Tables 1-2 for blocking a transport of a halide ion across a calcium activated chloride channel (CaCC), comprising contacting the CaCC with an effective amount of a compound of formula I, II, III, or IV or compounds of Tables 1-2 or a composition comprising a compound of formula I, II, III, or IV or compounds of Tables 1-2.

In another embodiment, this invention provides use of a compound of formula I, II, III or IV or compounds of Tables 1-2 or a composition comprising a compound of formula I, II, III, or IV or compounds of Tables 1-2 in the manufacture of a medicament for treating a disease responsive to blocking of a calcium activated chloride channel.

In another embodiment, this invention provides use of a compound of formula I, II, III, or IV or compounds of Tables 1-2 or a composition comprising a compound of formula I. II, III, or IV or compounds of Tables 1-2 in the manufacture of a medicament for blocking a transport of a halide ion across a calcium activated chloride channel.

The compounds and compositions can be administered alone or combined with other suitable therapy such as Oral Rehydration Therapy (ORT), supportive renal therapy, administration of an antiviral, vaccine, or other compound to treat the underlying infection or by administering an effective amount of an oral glucose-electrolyte solution to the animal. In another aspect, the compounds or compositions are co-administered with micronutrients, e.g., zinc, iron, and vitamin A. The therapies may be administered simultaneously or concurrently. Administration is by any appropriate route and varies with the disease or disorder to be treated and the acre and general health of the animal or human patient.

The compounds described herein can be administered on a mucosal surface of the gastrointestinal tract (e.g., by an enteral route, such as oral, intraintestinal, intraluminally, rectal as a suppository, and the like) or to a mucosal surface of the oral or nasal cavities (e.g., intranasal, buccal, sublingual, and the like) or to lungs. In one embodiment, the compounds disclosed herein are administered in a pharmaceutical formulation suitable for oral administration, intraluminally or intraperitoneal administration, or via inhalation therapy. In another embodiment, the compounds disclosed herein are administered in a pharmaceutical formulation suitable for sustained release.

In some embodiments of the methods of the invention, the composition is administered by a parenteral route. In some embodiments, the parenteral route includes, but is not limited to, intravenous, intramuscular, intraperitoneal and subcutaneous administration. In some embodiments of the methods of the invention, the composition is administered by an oral route. In some embodiments, the composition is formulated for oral administration in a formulation including, but not limited to, capsules, tablets, elixirs, suspensions and syrups. In some embodiments of the methods of the invention, the composition is formulated as a controlled release formulation. In some embodiments of the methods of the invention, the composition is administered in combination with a second agent for the treatment of the disease. In some embodiments, the second agent includes, but is not limited to, expectorants, mucolytics, antibiotics, anti-histamines, steroids, anti-inflammatory agents, and decongestants.

In one aspect, the compound is administered in a sustained release formulation which comprises the compound and an effective amount of a pharmaceutically-acceptable polymer. Such sustained release formulations provide a composition having a modified pharmacokinetic profile that is suitable for treatment as described herein. In one aspect of the invention, the sustained release formulation provides decreased Cₘₐₓ and increased Tₘₐₓ without altering bioavailability of the drug.

In one aspect, the compound is admixed with about 0.2% to about 5.0% wily solution of a pharmaceutically-acceptable polymer, in other embodiments, the amount of pharmaceutically-acceptable polymer is between about 0.25% and about 5.0%; between about 1% and about 4.5%; between about 2.0% and about 4.0%; between about 2.5% and about 3.5%; or alternatively about 0.2%; about 0.25%; about 0.3%; about 0.35%; about 0.4%; about 0.45%; about 0.5%, about 1.0%, about 2.0%, about 3.0%, or about 4.0%, of the polymer.

The therapeutic and prophylactic methods of this invention are useful to treat human patients in need of such treatment. However, the methods are not to be limited only to human patient but rather can be practiced and are intended to treat any animal in need thereof. Such animals will include, but not be limited to farm animals and pets such as simians, cows, pigs and horses, sheep, goats, cats and dogs. Diarrhea, also known as scours, is a cause of death in these animals. Infections with rotavirus and coronavirus are common in newborn calves and pigs. Rotavirus infection often occurs within 12 hours of birth. Symptoms of rotaviral infection include excretion of watery feces, dehydration and weakness. Coronavirus which causes a more severe illness in the newborn animals, has a higher mortality rate than rotaviral infection. Often, however, a young animal may be infected with more than one virus or with a combination of viral and bacterial microorganisms at one time. This may increase the severity of the disease.

The methods can be practiced in vivo in an acceptable animal model to confirm in vitro efficacy or to treat the disease or condition as described above.

In one embodiment, the halide ion is at least one of I⁻, Cl⁻, or Br⁻. In one preferred embodiment, the halide ion is Cl⁻. In one embodiment, the mammalian cell is epithelial cell, bipolar cell, smooth muscle cell, acinar and duct cell of lachrymal, parotid, submandibular, and/or sublingual gland, endothelial cell, or kidney cell.

When used to treat or prevent the diseases responsive to blocking of CaCC, the compounds of the present invention can be administered singly, as mixtures of one or more compounds of the invention, or in mixture or combination with other agents useful for treating such diseases and/or the symptoms associated with such diseases. The compounds of the present invention may also be administered in mixture or in combination with agents useful to treat other disorders or maladies, such as steroids, membrane stabilizers, 5-lipoxygenase (5LO) inhibitors, leukotriene synthesis and receptor inhibitors, inhibitors of IgE isotype switching or IgE synthesis, IgG isotype switching or IgG synthesis, β-agonists, tryptase inhibitors, aspirin, cyclooxygenase (COX) inhibitors, methotrexate, anti-TNF drugs, retuxin, PD4 inhibitors, p38 inhibitors, PDE4 inhibitors, and antihistamines, to name a few. The compounds of the invention can be administered per se in the form of prodrugs or as pharmaceutical compositions, comprising an active compound or prodrug.

The method can be practiced in vitro or in vivo. When practiced in vitro, the method can be used to screen for compounds, compositions and methods that possess the same or similar activity using the methods provided in the accompanying examples. Activity is determined using the methods described herein or others known to those of skill in the art.

### C. Compounds of the Invention

Provided herein are methods using 1,3,4-oxadiazole-containing compounds as CaCC inhibitors. In one aspect, the method comprises a compound of formula I:

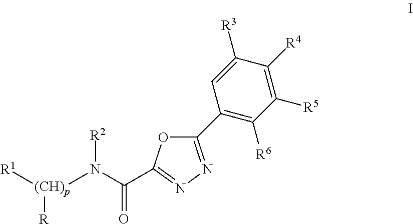

wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² together with the atoms bound thereto, form a heterocyclic or substituted heterocyclic ring;
R³ and R⁴, and R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;

or a pharmaceutically acceptable salt, isomer, or tautomer thereof.

In one aspect, the method comprises a compound of formula I:

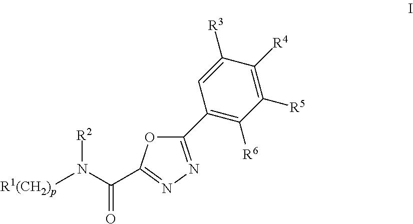

wherein
p is 0, 1, 2, or 3;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² together with the atoms bound thereto, form a heterocyclic or substituted heterocyclic ring;
R³ and R⁴, and R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;

or a pharmaceutically acceptable salt, isomer, or tautomer thereof.

In another aspect, the method comprises a prodrug of a compound of formula I.

Some embodiments of the compounds used in the methods of the present invention, are as described below.

In some embodiments, p is 0 or 1. In some embodiments, p is 0. In some embodiments, p is 1. In some embodiments, p is 2. In some embodiments, p is 3.

In some embodiments, R is hydrogen or methyl

In some embodiments, R⁶ hydrogen. In some embodiments, each of R³ and R⁵ is independently halo and R⁴ is hydrogen or hydroxyl. In some embodiments, R⁴ is hydroxyl.

In some embodiments, R² is hydrogen or methyl.

In some embodiments, each of R³, R⁵, and R⁶ is hydrogen; and R⁴ is sulfonylamino.

In some embodiments, each of R³, R⁴, and R⁶ is hydrogen; and R⁵ is sulfonylamino,

in some embodiments, R¹ and R² are taken together with the nitrogen atom to which they are bonded to form a heterocycle or substituted heterocycle. In some embodiments, when R¹ and R² are taken together with the nitrogen atom to which they are bonded to form a heterocycle or substituted heterocycle, the substituted heterocycle is substituted with alkyl, substituted alkyl, aryl or substituted aryl. In such embodiments, substituted alkyl is substituted with aryl. In such embodiments, substituted aryl is substituted with halo substituted alkyl.

In some embodiments of the above noted aspect, R¹ is alkyl, substituted alkyl, aryl, or substituted aryl. In some embodiments of R¹, substituted alkyl is substituted with aryl.

In some embodiments of the foregoing embodiment, substituted aryl is substituted with halo, alkyl, substituted alkyl, alkoxy, substituted alkoxy, aryloxy, or aryl. In some embodiments, substituted alkyl is substituted with halo or aryl. In some embodiments, substituted alkoxy is substituted with halo or aryl.

In some embodiments, the method comprises a compound formula II:

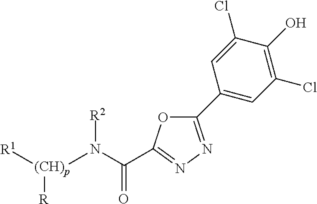

wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy; and
R² is selected from the group consisting of hydrogen, alkyl, substituted alkyl, alkenyl, substituted alkenyl, alkynyl, and substituted alkynyl;
   or R¹ and R² together with the atoms bound thereto, form a heterocyclic or substituted heterocyclic ring;
   or a pharmaceutically acceptable salt, isomer, or tautomer thereof.

In some embodiments, the method comprises a compound of formula II:

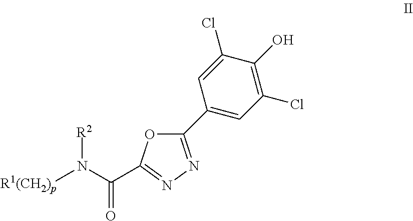

wherein
p is 0, 1, 2, or 3;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy; and
R² is selected from the group consisting of hydrogen, alkyl, substituted alkyl, alkenyl, substituted alkenyl, alkynyl, and substituted alkynyl;
   or R¹ and R² together with the atoms bound thereto, form a heterocyclic or substituted heterocyclic ring;

or a pharmaceutically acceptable salt, isomer, or tautomer thereof.

In some embodiments, p is 0 or 1. In some embodiments, p is 0. In some embodiments, p is 1. In some embodiments, p is 2. In some embodiments, p is 3.

In some embodiments, R is hydrogen or methyl.

In some embodiments, R² is hydrogen or methyl. In some embodiments, R² is hydrogen.

In some embodiments, p is 1 and R¹ is substituted alkyl or substituted aryl. In some embodiments, p is 1 and R¹ is substituted aryl. In some embodiments, R¹ is substituted aryl substituted with halo, alkyl, substituted alkyl, aryloxy, substituted alkoxy, or aryl. In some embodiments. R¹ is substituted phenyl. In some embodiments. R¹ is substituted alkyl substituted with aryl.

In some embodiments of the compound of formula II, p is 0 or 1; R is hydrogen or methyl; R¹ is substituted alkyl or substituted aryl; and R² is hydrogen or methyl.

In some embodiments of the compound of formula II, p is 0 or 1; R is hydrogen or methyl; R¹ is substituted alkyl substituted with aryl or substituted aryl substituted with halo, alkyl, substituted alkyl, aryloxy, substituted alkoxy, or aryl; and R² is hydrogen or methyl.

In some embodiments, R¹ and R² together with the atoms bound thereto, form a heterocyclic or a substituted heterocyclic ring. In some embodiments, the substituted heterocyclic ring is a substituted piperidine or a substituted piperazine.

In some embodiments, the method comprises a compound of formula I as described above, wherein each of R³, R⁴, and R⁶ is hydrogen. In yet another aspect, R⁵ is sulfonylamino.

In one aspect, the method comprises a compound of formula III:

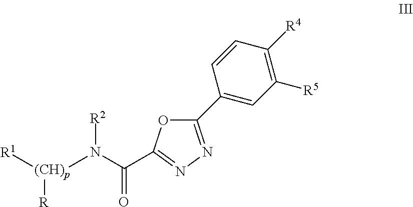

wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² together with the atoms bound thereto, for a heterocyclic or substituted heterocyclic ring; and
R⁴ and R⁵ are each independently selected from the group consisting of hydrogen and sulfonylamino;

or a pharmaceutically acceptable salt, isomer, or tautomer thereof.

In one aspect, the method comprises a compound of formula IIIa:

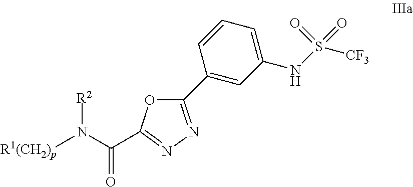

wherein
p is 0, 1, 2, or 3;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy; and
R² is selected from the group consisting of hydrogen, alkyl, substituted alkyl, alkenyl, substituted alkenyl, alkynyl, and substituted alkynyl;
   or R¹ and R² together with the atoms bound thereto, form a heterocyclic or substituted heterocyclic ring;

or a pharmaceutically acceptable salt, isomer, or tautomer thereof

In one aspect, the method comprises a compound of formula IIIb;

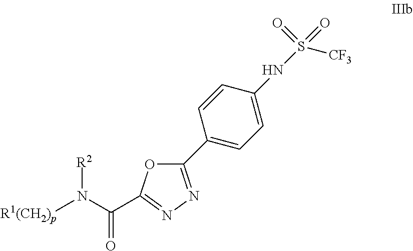

wherein
p is 0, 1, 2, or 3;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy; and
R² is selected from the group consisting of hydrogen, alkyl, substituted alkyl, alkenyl, substituted alkenyl, alkynyl, and substituted alkynyl;
   or R¹ and R² together with the atoms bound thereto, form a heterocyclic or substituted heterocyclic ring;

or a pharmaceutically acceptable salt, isomer, or tautomer thereof.

In some embodiments of the foregoing aspects, R is hydrogen or methyl.

In some embodiments of the foregoing aspects, p is 0 or 1. In some embodiments, p is 0. In some embodiments, p is 1. In some embodiments, p is 2. In some embodiments, p is 3.

In some embodiments of the foregoing aspects R² is hydrogen or methyl. In some embodiments of the foregoing aspects, p is 1 and R¹ is aryl or substituted aryl. In some embodiments of the foregoing aspects, p is 1 and R¹ is substituted aryl. In some embodiments of the foregoing aspects, R¹ is substituted phenyl. In some embodiments of the foregoing aspects, R¹ is substituted aryl substituted with halo, alkyl, substituted alkyl, or aryloxy.

In some embodiments of the foregoing aspects, p is 0 or 1; R is hydrogen or methyl; R¹ is aryl or substituted aryl substituted with halo, alkyl, substituted alkyl, or aryloxy; and R² is hydrogen or methyl.

In some embodiments of the foregoing aspects, R⁴ and R⁵ are independently selected from the group consisting of hydrogen or sulfonylamino.

In some embodiments of the foregoing aspects, p is 0 or 1; R is hydrogen or methyl; R¹ is aryl or substituted aryl; R² is hydrogen or methyl; R⁴ is hydrogen; and R⁵ is sulfonylamino.

In some embodiments of the foregoing aspects, p is 0 or 1; R is hydrogen or methyl; R¹ is aryl or substituted aryl; R² is hydrogen or methyl; R⁵ is hydrogen; and R⁴ is sulfonylamino,

In some embodiments of the foregoing aspects, R¹ and R² together with the atoms bound thereto, form a heterocyclic or a substituted heterocyclic ring. In some embodiments of the foregoing aspects, the substituted heterocyclic ring is a substituted piperidine or a substituted piperazine.

In one aspect, the method comprises a compound of formula IV:

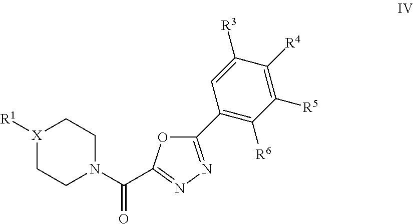

wherein
X is CH or N; and
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R³ and R⁴, and R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;

or a pharmaceutically acceptable salt, isomer, or tautomer thereof.

In some embodiments, X is CH.

in some embodiments, X is N.

In some embodiments, R⁶ is hydrogen.

In some embodiments, each of R³ and R⁵ is independently halo; and R⁴ is hydroxyl.

In some embodiments, each of R³, R⁴, and R⁶ is hydrogen; and R⁵ is sulfonylamino.

In some embodiments, each of R³, R⁵, and R⁶ is hydrogen; and R⁴ is sulfonylamino.

In some embodiments, R¹ is alkyl, substituted alkyl, aryl, or substituted aryl.

In some embodiments of the compound of formula IV, X is CH; each of R³ and R⁵ is independently halo; R⁴ is hydroxyl; R⁶ is hydrogen; and R¹ is alkyl, substituted alkyl, aryl, or substituted aryl.

In some embodiments of the compound of formula IV, X is N; each of R³ and R⁵ is independently halo; R⁴ is hydroxyl; R⁶ is hydrogen; and R¹ is alkyl, substituted alkyl, aryl, or substituted aryl.

In some embodiments of the compound of formula IV, X is CH; each of R³, R⁵, and R⁶ is hydrogen; R⁵ is sulfonylamino; and R¹ is alkyl, substituted alkyl, aryl, or substituted aryl.

In some embodiments of the compound of formula IV, X is N; each of R³, R⁴, and R⁶ is hydrogen; R⁵ is sulfonylamino; and R¹ is alkyl, substituted alkyl, aryl, or substituted aryl.

In some embodiments of the compound of formula IV, X is CH; each of R³, R⁵, and R⁶ is hydrogen; R⁴ is sulfonylamino; and R¹ is alkyl, substituted alkyl, aryl, or substituted aryl.

In some embodiments of the compound of formula IV, X is N; each of R³, R⁵, and R⁶ is hydrogen; R⁴ is sulfonylamino; and R¹ is alkyl, substituted alkyl, aryl, or substituted aryl.

In some aspects of the foregoing embodiments, substituted alkyl is substituted with aryl. In some aspects of the foregoing embodiments, substituted aryl is substituted with substituted alkyl.

In another embodiment, the method comprises a compound selected from the group consisting of:
5-(3,5-dichloro-4-hydroxyphenyl)-N-(3-(trifluoromethoxy)benzyl)-1,3,4-oxadiazole-2-carboxamide;
5-(3,5-dichloro-4-hydroxyphenyl)-N-(4-phenoxybenzyl)-1,3,4-oxadiazole-2-carboxamide;
(4-benzylpiperidin-1-yl)(5-(3,5-dichloro-4-hydroxyphenyl)-1,3,4-oxadiazol-2-yl)methanone;
(5-(3,5-dichloro-4-hydroxyphenyl)-1,3,4-oxadiazol-2-yl)(4-(3-(trifluoromethyl)phenyl)piperazin-1-yl)methanone;
N-(4-tert-butyl benzyl)-5-(3,5-dichloro-4-hydroxyphenyl)-1,3,4-oxadiazole-2-carboxamide;
N-benzhydryl-5-(3,5-dichloro-4-hydroxyphenyl)-1,3,4-oxadiazole-7-carboxamide;
N-(4-phenoxybenzyl)-5-(3-(trifluoromethylsulfonamido)phenyl)-1,3,4-oxadiazole-2-carboxamide;
N-(3-(5-(4-benzylpiperidine-1-carbonyl)-1,3,4-oxadiazol-2-yl)phenyl)-1,1,1-trifluoromethanesulfonamide;
N-(4-tert-butylbenzyl)-5-(3-(trifluoromethylsulfonamido)phenyl)-1,3,4-oxadiazole-2-carboxamide;
N-(3,4-dichlorobenzyl)-N-methyl-5-(3-(trifluoromethylsulfonamido)phenyl)-1,3,4-oxadiazole-2-carboxamide;
1,1,1-trifluoro-N-(3-(5-(4-(3-(trifluoromethyl)phenyl)piperazine-1-carbonyl)-1,3,4-oxadiazol-2-yl)phenyl)methanesulfonamide;
5-(3,5-dichloro-4-hydroxyphenyl)-N-(3,4-dichlorobenzyl)-N-methyl-1,3,4-oxadiazole-2-carboxamide;
5-(3,5-dichloro-4-hydroxyphenyl)-N-methyl-N-(3-phenoxybenzyl)-1,3,4-oxadiazole-2-carboxamide;
5-(3,5-dichloro-4-hydroxyphenyl)-N-(3-phenoxybenzyl)-1,3,4-oxadiazole-2-carboxamide;
5-(3,5-dichloro-4-hydroxyphenyl)-N-(2,2-diphenylethyl)-1,3,4-oxadiazole-2-carboxamide;
N-(3-(benzyloxy)benzyl)-5-(3,5-dichloro-4-hydroxyphenyl)-1,3,4-oxadiazole-2-carboxamide;
N-(3,4-dichlorobenzyl)-N-methyl-5-(4-(trifluoromethylsulfonamido)phenyl)-1,3,4-oxadiazole-2-carboxamide;
N-(4-(benzyloxy)benzyl)-5-(3,5-dichloro-4-hydroxyphenyl)-1,3,4-oxadiazole-2-carboxamide;
N-(biphenyl-3-ylmethyl)-5-(3,5-dichloro-4-hydroxyphenyl)-1,3,4-oxadiazole-2-carboxamide;
N-(4-tert-butylbenzyl)-5-O-(trifluoromethylsulfonamido)phenyl)-1,3,4-oxadiazole-2-carboxamide;
5-(3,5-dichloro-4-hydroxyphenyl)-N-(3-fluoro-5-(trifluoromethyl)benzyl)-1,3,4-oxadiazole-2-carboxamide;
5-(3,5-dichloro-4-hydroxyphenyl)-N-(4-(trifluoromethoxy)benzyl)-1,3,4-oxadiazole-2-carboxamide;
N-(4-phenoxybenzyl)-5-(4-(trifluoromethylsulfonamido)phenyl)-1,3,4-oxadiazole-2-carboxamide;
5-(3,5-dichloro-4-hydroxyphenyl)-N-(4-fluoro-3-(trifluoromethyl)benzyl)-1,3,4-oxadiazole-2-carboxamide;
1,1,1-trifluoro-N-(4-(5-(4-(3-(trifluoromethyl)phenyl)piperazine-1-carbonyl)-1,3,4-oxadiazol-2-yl)phenyl)methanesulfonamide, and
N-(1-(4-chlorophenyl)ethyl)-5-(3,5-dichloro-4-hydroxyphenyl)-1,3,4-oxadiazole-2-carboxamide, or
a pharmaceutically acceptable salt, isomer, or tautomer thereof.

it will be appreciated by one of skill in the an that the embodiments summarized above may be used together in any suitable combination to generate additional embodiments not expressly recited above, and that such embodiments are considered to be part of the present invention.

Those of skill in the art will appreciate that the compounds described herein may include functional groups that can be masked with progroups to create prodrugs. Such prodrugs are usually, but need not be, pharmacologically inactive until converted into their active drug form. The compounds described in this invention may include promoieties that are hydrolyzable or otherwise cleavable under conditions of use. For example, ester groups commonly undergo acid-catalyzed hydrolysis to yield the parent hydroxyl group when exposed to the acidic conditions of the stomach or base-catalyzed hydrolysis when exposed to the basic conditions of the intestine or blood. Thus, when administered to a subject orally, compounds that include ester moieties can be considered prodrugs of their corresponding hydroxyl, regardless of whether the ester form is pharmacologically active.

Prodrugs designed to cleave chemically in the stomach to the active compounds can employ progroups including such esters. Alternatively, the progroups can be designed to metabolize in the presence of enzymes such as esterases, amidases, lipolases, and phosphatases, including ATPases and kinase, etc. Progroups including linkages capable of metabolizing in vivo are well known and include, by way of example and not limitation, ethers, thioethers, silylethers, silylthioethers, esters, thioesters, carbonates, thiocarbonates, carbamates, thiocarbamates, ureas, thioureas, and carboxamides.

In the prodrugs, any available functional moiety can be masked with a progroup to yield a prodrug. Functional groups within the compounds of the invention that can be masked with progroups include, but are not limited to, amines (primary and secondary), hydroxyls, sulfanyls (thiols), and carboxyls. A wide variety of progroups suitable for masking functional groups in active compounds to yield prodrugs are well-known in the art. For example, a hydroxyl functional group can be masked as a sulfonate, ester, or carbonate promoiety, which can be hydrolyzed in vivo to provide the hydroxyl group. An amino functional group can be masked as an amide, carbamate, imine, urea, phosphenyl, phosphoryl, or sulfenyl promoiety, which can be hydrolyzed in vivo to provide the amino group. A carboxyl group can be masked as an ester (including silyl esters and thioesters), amide, or oxadiazolepromoiety, which can be hydrolyzed in vivo to provide the carboxyl group. Other specific examples of suitable progroups and their respective promoieties will be apparent to those of skill in the art. All of these progroups, alone or in combinations, can be included in the prodrugs.

As noted above, the identity of the progroup is not critical, provided that it can be metabolized under the desired conditions of use, for example, under the acidic conditions found in the stomach and/or by enzymes found in vivo, to yield a biologically active group, e.g., the compounds as described herein. Thus, skilled artisans will appreciate that the progroup can comprise virtually any known or later-discovered hydroxyl, amine or thiol protecting group. Non-limiting examples of suitable protecting groups can be found, for example, in PROTECTIVE GROUPS IN ORGANIC SYNTHESIS, Greene & Wuts, 2nd Ed., John Wiley & Sons, New York, 1991.

Additionally, the identity of the progroup(s) can also be selected so as to impart the prodrug with desirable characteristics. For example, lipophilic groups can be used to decrease water solubility and hydrophilic groups can be used to increase water solubility. In this way, prodrugs specifically tailored for selected modes of administration can be obtained. The progroup can also be designed to impart the prodrug with other properties, such as, for example, improved passive intestinal absorption, improved transport-mediated intestinal absorption, protection against fast metabolism (slow-release prodrugs), tissue-selective delivery, passive enrichment in target tissues, and targeting-specific transporters. Groups capable of imparting prodrugs with these characteristics are well-known and are described, for example, in Ettmayer et al. (2004), J. Med. Chem. 47 (10):2393-2404. All of the various groups described in these references can be utilized in the prodrugs described herein.

As noted above, progroup(s) may also be selected to increase the water solubility of the prodrug as compared to the active drug. Thus, the progroup(s) may include or can be a group(s) suitable for imparting drug molecules with improved water solubility. Such groups are well-known and include, by way of example and not limitation, hydrophilic groups such as alkyl, aryl, and arylalkyl, or cycloheteroalkyl groups substituted with one or more of an amine, alcohol, a carboxylic acid, a phosphorous acid, a sulfoxide, a sugar, an amino acid, a thiol, a polyol, an ether, a thioether, and a quaternary amine salt. Numerous references teach the use and synthesis of prodrugs, including, for example. Ettmayer et al., supra and Bungaard et al. (1989) J. Med. Chem. 32 (12): 2503-2507.

One of ordinary skill in the art will appreciate that many of the compounds of the invention and prodrugs thereof, may exhibit the phenomena of tautomerism, conformational isomerism, geometric isomerism, and/or optical isomerism. For example, the compounds and prodrugs of the invention may include one or more chiral centers and/or double bonds and as a consequence may exist as stereoisomers, such as double-bond isomers (i.e., geometric isomers), enantiomers, diasteromers, and mixtures thereof, such as racemic mixtures. As another example, the compounds and prodrugs of the invention may exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, it should be understood that the invention encompasses any tautomeric, conformational isomeric, optical isomeric, and/or geometric isomeric forms of the compounds or prodrugs having one or more of the utilities described herein, as well as mixtures of these various different isomeric forms.

Depending upon the nature of the various substituents, the compounds and prodrugs of the invention can be in the form of salts. Such salts include pharmaceutically acceptable salts, salts suitable for veterinary uses, etc. Such salts can be derived from acids or bases, as is well-known in the art. In one embodiment, the salt is a pharmaceutically acceptable salt.

in one embodiment, the compound, isomer, tautomer, prodrug, or pharmaceutically acceptable salt thereof, is selected from Tables 1-2.

TABLE 1
I

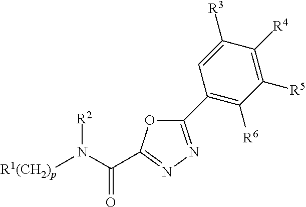

| Cmpd No. | N(R²)(CH₂)ₚR¹ | R³ | R⁴ | R⁵ | R⁶ |
|---|---|---|---|---|---|
| 1 | 3-trifluomethoxybenzylamino | Cl | OH | Cl | H |
| 2 | 4-phenoxybenzylamino | Cl | OH | Cl | H |
| 3 | 4-benzylpiperidin-1-yl | Cl | OH | Cl | H |
| 4 | 4-(3-trifluoromethylphenyl)piperazin-1-yl | Cl | OH | Cl | H |
| 5 | 4-tert-butylbenzylamino | Cl | OH | Cl | H |
| 6 | diphenylmethylamino | Cl | OH | Cl | H |
| 7 | 4-phenoxybenzylamino | H | H | NHSO₂CF₃ | H |
| 8 | 4-benzylpiperidin-1-yl | H | H | NHSO₂CF₃ | H |
| 9 | 4-tert-butylbenzylamino | H | H | NHSO₂CF₃ | H |
| 10 | N-methyl-3,4-dichlorobenzylamino | H | H | NHSO₂CF₃ | H |
| 11 | 4-(3-trifluoromethylphenyl)piperazin-1-yl | H | H | NHSO₂CF₃ | H |
| 12 | N-methyl-3,4-dichlorobenzylamino | Cl | OH | Cl | H |
| 13 | N-methyl-3-phenoxybenzylamino | Cl | OH | Cl | H |
| 14 | 3-phenoxybenzylamino | Cl | OH | Cl | H |
| 15 | 2,2-diphenylethylamino | Cl | OH | Cl | H |
| 16 | 3-(benzyloxy)benzylamino | Cl | OH | Cl | H |
| 17 | N-methyl-3,4-dichlorobenzylamino | H | NHSO₂CF₃ | H | H |
| 18 | 4-(benzyloxy)benzylamino | Cl | OH | Cl | H |
| 19 | biphenyl-3-ylmethylamino | Cl | OH | Cl | H |
| 20 | 4-tert-butylbenzylamino | H | NHSO₂CF₃ | H | H |
| 21 | 3-fluoro-5-(trifluoromethyl)benzylamino | Cl | OH | Cl | H |
| 22 | 4-(trifluoromethoxy)benzylamino | Cl | OH | Cl | H |
| 23 | 4-phenoxybenzylamino | H | NHSO₂CF₃ | H | H |
| 24 | 4-fluoro-3-(trifluoromethyl)benzylamino | Cl | OH | Cl | H |
| 25 | 4-(3-(trifluoromethyl)phenyl)piperazin-1-yl | H | NHSO₂CF₃ | H | H |
| 26 | 1-(4-chlorophenyl)ethanamino | Cl | OH | Cl | H |

Table 2 lists the structures and names of compounds listed in Table 1.

TABLE 2

| Cmpd No. | Structure | Name |
|---|---|---|
| 1 | 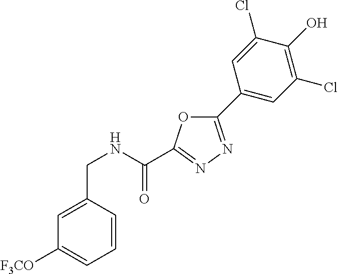 | 5-(3,5-dichloro-4- hydroxyphenyl)-N-(3- (trifluoromethoxy) benzyl)-1,3,4- oxadiazole- 2-carboxamide, |
| 2 | 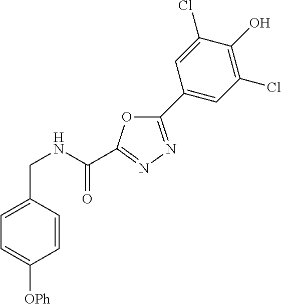 | 5-(3,5-dichloro-4- hydroxyphenyl)-N-(4- phenoxybenzyl)-1,3,4- oxadiazole-2- carboxamide |
| 3 | 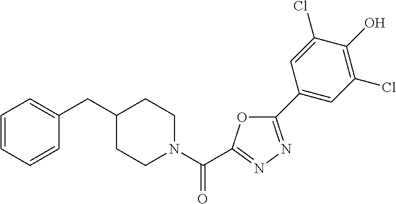 | (4-benzylpiperidin-1- yl)(5-(3,5-dichloro-4- hydroxyphenyl)-1,3,4- oxadiazol-2- yl)methanone |
| 4 | 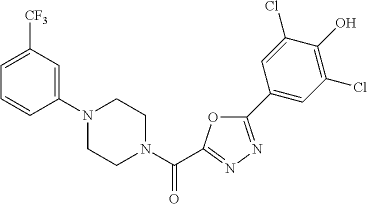 | (5-(3,5-dichloro-4- hydroxyphenyl)-1,3,4- oxadiazol-2-yl)(4-(3- (trifluoromethyl) phenyl)piperazin-1- yl)methanone |
| 5 | 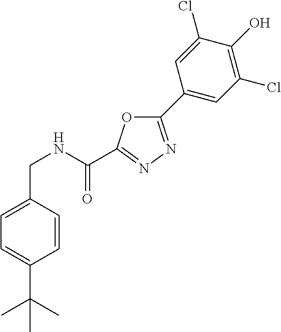 | N-(4-tert- butylbenzyl)-5-(3,5- dichloro-4- hydroxyphenyl)-1,3,4- oxadiazole-2- carboxamide |
| 6 | 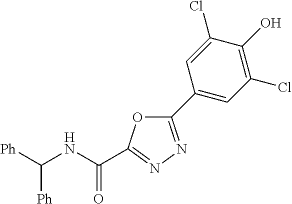 | N-benzhydryl-5-(3,5- dichloro-4- hydroxyphenyl)-1,3,4- oxadiazole-2- carboxamide |
| 7 | 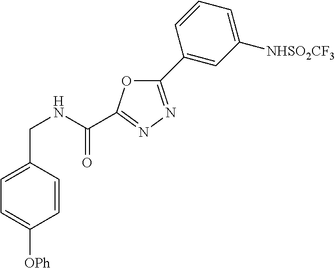 | N-(4-phenoxybenzyl)- 5-(3- (trifluoromethyl- sulfonamido) phenyl)-1,3,4- oxadiazole-2- carboxamide |
| 8 | 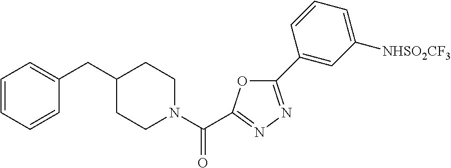 | N-(3-(5-(4- benzylpiperidine-1- carbonyl)-1,3,4- oxadiazol-2- yl)phenyl)-1,1,1- trifluoromethane- sulfonamide |
| 9 | 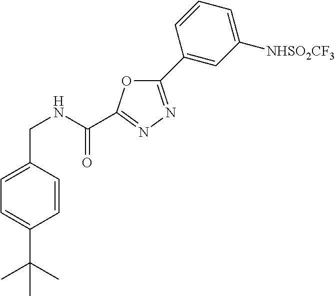 | N-(4-tert- butylbenzyl)-5-(3- (trifluoromethyl- sulfonamido) phenyl)-1,3,4- oxadiazole-2- carboxamide |
| 10 | 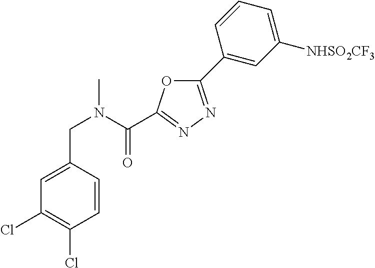 | N-(3,4- dichlorebenzyl)-N- methyl-5-(3- (trifluoromethyl- sulfonamido) phenyl)-1,3,4- oxadiazole-2- carboxamide |
| 11 | 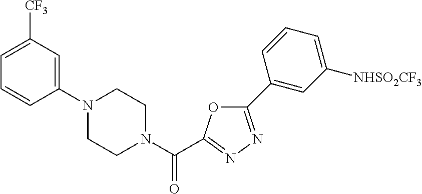 | 1,1,1-trifluoro-N-(3- (5-(4-(3- (trifluoromethyl) phenyl)piperazine- 1-carbonyl)-1,3,4- oxadiazol-2- yl)phenyl)methane- sulfonamide |
| 12 | 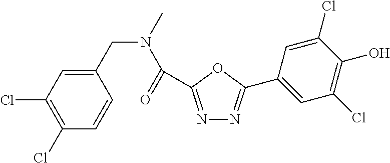 | 5-(3,5-dichloro-4- hydroxyphenyl)-N- (3,4-dichlorobenzyl)- N-methyl-1,3,4- oxadiazole-2- carboxamide |
| 13 | 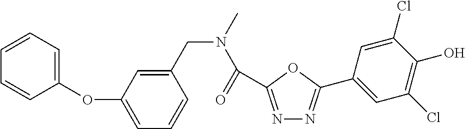 | 5-(3,5-dichloro-4- hydroxyphenyl)-N- methyl-N-(3- phenoxybenzyl)-1,3,4- oxadiazole-2- carboxamide |
| 14 | 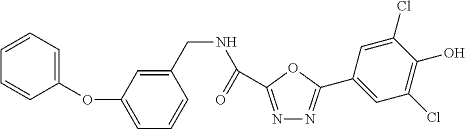 | 5-(3,5-dichloro-4- hydroxyphenyl)-N-(3- phenoxybenzyl)-1,3,4- oxadiazole-2- carboxamide |
| 15 | 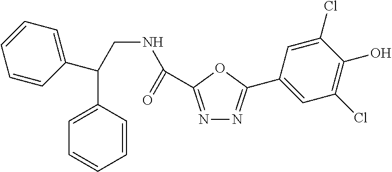 | 5-(3,5-dichloro-4- hydroxyphenyl)-N- (2,2-diphenylethyl)- 1,3,4-oxadiazole-2- carboxamide |
| 16 | 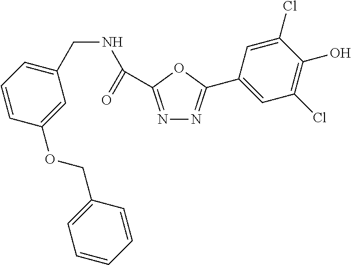 | N-(3- (benzyloxy)benzyl)-5- (3,5-dichloro-4- hydroxyphenyl)-1,3,4- oxadiazole-2- carboxamide |
| 17 | 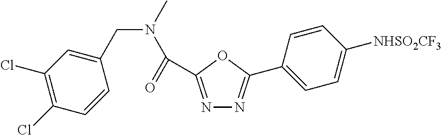 | N-(3,4- dichlorobenzyl)-N- methyl-5-(4- (trifluoromethyl- sulfonamido) phenyl)-1,3,4- oxadiazole-2- carboxamide |
| 18 | 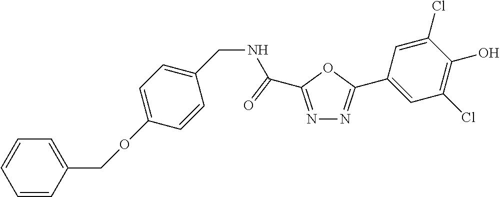 | N-(4- (benzyloxy)benzyl)- 5-(3,5-dichloro-4- hydroxyphenyl)-1,3,4- oxadiazole-2- carboxamide |
| 19 | 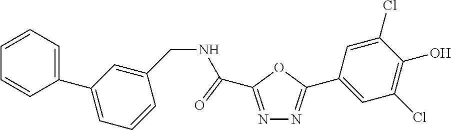 | N-(biphenyl-3- ylmethyl)-5-(3,5- dichloro-4- hydroxyphenyl)- 1,3,4-oxadiazole-2- carboxamide |
| 20 | 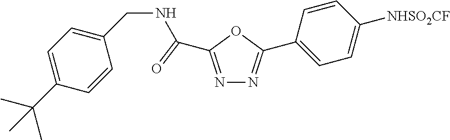 | N-(4-tert- butylbenzyl)-5-(4- (trifluoromethyl- sulfonamido)phenyl)- 1,3,4-oxadiazole-2- carboxamide |
| 21 | 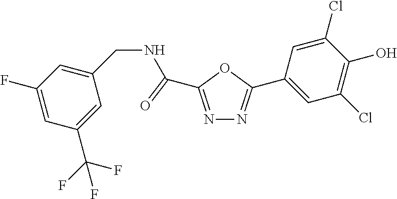 | 5-(3,5-dichloro-4- hydroxyphenyl)-N- (3-fluoro-5- (trifluoromethyl) benzyl)-1,3,4- oxadiazole-2- carboxamide |
| 22 | 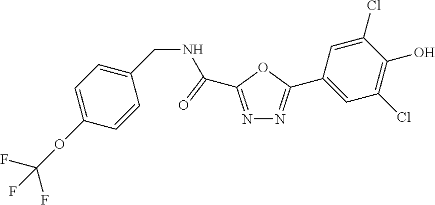 | 5-(3,5-dichloro-4- hydroxyphenyl)-N-(4- (trifluoromethoxy) benzyl)-1,3,4- oxadiazole- 2-carboxamide |
| 23 | 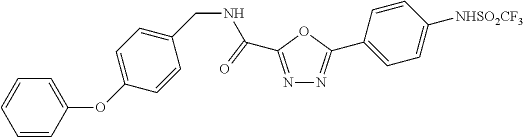 | N-(4-phenoxybenzyl)- 5-(4- (trifluoromethyl- sulfonamido) phenyl)-1,3,4- oxadiazole-2- carboxamide |
| 24 | 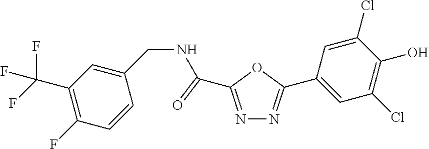 | 5-(3,5-dichloro-4- hydroxyphenyl)-N- (4-fluoro-3- (trifluoromethyl) benzyl)-1,3,4- oxadiazole-2- carboxamide |
| 25 | 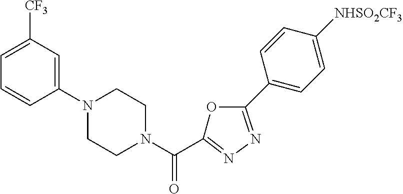 | 1,1,1-trifluoro-N-(4- (5-(4-(3- (trifluoromethyl) phenyl)piperazine- 1-carbonyl)-1,3,4- oxadiazol-2- yl)phenyl)methane- sulfonamide |
| 26 | 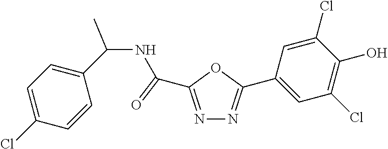 | N-(1-(4- chlorophenyl)ethyl)-5- (3,5-dichloro-4- hydroxyphenyl)- 1,3,4-oxadiazole-2- carboxamide |

### D. Pharmaceutical Formulations and Administration

The compounds or isomers, prodrug, tautomer, or pharmaceutically acceptable salts thereof, of the present invention can be formulated in the pharmaceutical compositions per se, or in the form of a hydrate, solvate. N-oxide, or pharmaceutically acceptable salt, as described herein. Typically, such salts are more soluble in aqueous solutions than the corresponding free acids and bases, but salts having lower solubility than the corresponding free acids and bases may also be formed. The present invention includes within its scope solvates of the compounds and salts thereof, for example, hydrates. The compounds may have one or more asymmetric centers and may accordingly exist both as enantiomers and as diastereoisomers. It is to be understood that all such isomers and mixtures thereof are encompassed within the scope of the present invention.

In one embodiment, the methods of the invention comprise administering a pharmaceutical composition comprising a compound provided herein and a pharmaceutically acceptable carrier. In another embodiment, the methods of the invention comprise administering a pharmaceutical composition comprising a therapeutically effective amount of a compound provided herein and a pharmaceutically acceptable carrier. In one embodiment, the methods of the invention comprise administering a pharmaceutical formulation comprising a compound selected from the compounds described herein, or isomers, hydrates, tautomer, or pharmaceutically acceptable salts thereof and at least one pharmaceutically acceptable excipient, diluent, preservative, stabilizer, or mixture thereof.

In one embodiment, the methods can be practiced as a therapeutic approach towards the treatment of the conditions described herein. Thus, in a specific embodiment, the compounds described herein can be used to treat the conditions described herein in animal subjects, including humans. The methods generally comprise administering to the subject an amount of a compound of the invention, or a salt, prodrug, hydrate, or N-oxide thereof, effective to treat the condition.

In some embodiments, the subject is a non-human mammal, including, but not limited to, bovine, horse, feline, canine, rodent, or primate. In another embodiment, the subject is a human.

The compounds described herein can be provided in a variety of formulations and dosages. It is to be understood that reference to the compound of the invention, or “active” in discussions of formulations is also intended to include, where appropriate as known to those of skill in the art, formulation of the salt, prodrug, hydrate, or N-oxide thereof of the compounds.

In one embodiment, the compounds are provided as non-toxic pharmaceutically acceptable salts. Suitable pharmaceutically acceptable salts of the compounds described herein include acid addition salts such as those formed with hydrochloric acid, fumaric acid, p-toluenesulphonic acid, maleic acid, succinic acid, acetic acid, citric acid, tartaric acid, carbonic acid, or phosphoric acid. Salts of amine groups may also comprise quaternary ammonium salts in which the amino nitrogen atom carries a suitable organic group such as an alkyl, alkenyl, alkynyl, or substituted alkyl moiety. Furthermore, where the compounds carry an acidic moiety, suitable pharmaceutically acceptable salts thereof may include metal salts such as alkali metal salts, e.g., sodium or potassium salts; and alkaline earth metal salts, e.g., calcium or magnesium salts.

The pharmaceutically acceptable salts described herein can be formed by conventional means, such as by reacting the free base form of the product with one or more equivalents of the appropriate acid in a solvent or medium in which the salt is insoluble or in a solvent such as water which is removed in vacuo, by freeze drying, or by exchanging the anions of an existing salt for another anion on a suitable ion exchange resin.

Pharmaceutical compositions comprising the compounds described herein (or prodrugs thereof) can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries which facilitate processing of the active compounds into preparations which can be used pharmaceutically.

The compounds described herein can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

The compounds provided herein are capable of crossing the blood brain barrier (BBB), making these compounds particularly useful in treating stroke, tumors or infections in the brain, or the spinal cord. It is known in the art that neutral L-amino acids have various rates of movement into the brain. Phenylalanine, leucine, tyrosine, isoleucine, valine, tryptophan, methionine, histidine and L-dihydroxy-phenylalanine (l-DOPA) may enter as rapidly as glucose. These essential amino acids may not be synthesized by the brain and, therefore, may be supplied from protein breakdown and diet. Alternatively, various pharmaceutically acceptable carriers, such as a nanoparticie as disclosed in Schroder and Sabel (1996) Brain Research 710 (1-2):121-124, or a blood brain barrier permeation peptide as disclosed in United States Patent Application Publication No.: 20060039859, are incorporated herein by reference in their entirety.

The pharmaceutical compositions for the administration of the compounds can be conveniently presented in dosage unit form and can be prepared by any of the methods well known in the art of pharmacy. The pharmaceutical compositions can be, for example, prepared by uniformly and intimately bringing the active ingredient into association with a liquid carrier, a finely divided solid carrier or both, and then, if necessary, shaping the product into the desired formulation. In the pharmaceutical composition, the active object compound is included in an amount sufficient to produce the desired therapeutic effect. For example, pharmaceutical compositions of the invention may take a form suitable for virtually any mode of administration, including, for example, topical, ocular, oral, buccal, systemic, nasal, injection, transdermal, rectal, and vaginal, or a form suitable for administration by inhalation or insufflation.

For topical administration, the compound(s) or prodrug(s) can be formulated as solutions, gels, ointments, creams, suspensions, etc., as is well-known in the art.

Systemic formulations include those designed for administration by injection (e.g., subcutaneous, intravenous, intramuscular, intrathecal, or intraperitoneal injection) as well as those designed for transdermal, transmucosal, oral, or pulmonary administration.

Useful injectable preparations include sterile suspensions, solutions, or emulsions of the active compound(s) in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the active compound(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium laurel sulfate). The tablets can be coated by methods well known in the art with, for example, sugars, films, or enteric coatings. Additionally, the pharmaceutical compositions are in a form suitable for oral use may also include, for example, troches, lozenges, aqueous, or oily suspensions, dispersible powders or granules, emulsions, hard or soft capsules, or syrups or elixirs.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the active ingredient (including drug and/or prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g., starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and 4,265,874 to form osmotic therapeutic tablets for control release. The pharmaceutical compositions described herein may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Preparations for oral administration can be suitably formulated to give controlled release or sustained release of the active compound, as is well known. The sustained release formulations of this invention are preferably in the form of a compressed tablet comprising an intimate mixture of compound described herein and a partially neutralized pH-dependent binder that controls the rate of compound dissolution in aqueous media across the range of pH in the stomach (typically approximately 2) and in the intestine (typically approximately about 5.5).

To provide for a sustained release of compounds described herein, one or more pH-dependent binders can be chosen to control the dissolution profile of the sustained release formulation so that the formulation releases compound slowly and continuously as the formulation is passed through the stomach and gastrointestinal tract. Accordingly, the pH-dependent binders suitable for use in this invention are those which inhibit rapid release of drug from a tablet during its residence in the stomach (where the pH is-below about 4.5), and which promotes the release of a therapeutic amount of the compound from the dosage form in the lower gastrointestinal tract (where the pH is generally greater than about 4.5). Many materials known in the pharmaceutical art as “enteric” binders and coating agents have a desired pH dissolution properties. The examples include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof. One or more pH-dependent binders present in the sustained release formulation of the invention are in an amount ranging from about 1 to about 20 wt %, more preferably from about 5 to about 12 wt % and most preferably about 10 wt %.

One or more pH-independent binders may be in used in oral sustained release formulation of the invention. The pH-independent binders can be present in the formulation of this invention in an amount ranging from about 1 to about 10 wt %, and preferably in amount ranging from about 1 to about 3 wt % and most preferably about 2 wt %.

The sustained release formulation of the invention may also contain pharmaceutical excipients intimately admixed with the compound and the pH-dependent binder. Pharmaceutically acceptable excipients may include, for example, pH-independent binders or film-forming agents such as hydroxypropyl methylcellulose, hydroxypropyl cellulose, methylcellulose, polyvinylpyrrolidone, neutral poly(meth)acrylate esters, starch, gelatin, sugars, carboxymethylcellulose, and the like. Other useful pharmaceutical excipients include diluents such as lactose, mannitol, dry starch, microcrystalline cellulose and the like; surface active agents such as polyoxyethylene sorbitan esters, sorbitan esters and the like; and coloring agents and flavoring agents. Lubricants (such as talc and magnesium stearate) and other tableting aids can also be optionally present.

The sustained release formulations of this invention have a compound described herein in the range of about 50% by weight to about 95% or more by weight, and preferably between about 70% to about 90% by weight; a pH-dependent binder content of between 5% and 40%, preferably between 5% and 25%, and more preferably between 5% and 15%; with the remainder of the dosage form comprising pH-independent binders, fillers, and other optional excipients.

For buccal administration, the compositions may take the form of tablets or lozenges formulated in the conventional manner.

For rectal and vaginal routes of administration, the active compound(s) can be formulated as solutions (for retention enemas), suppositories, or ointments containing conventional suppository bases such as cocoa butter or other glycerides.

For nasal administration or administration by inhalation or installation, the active compound(s) or prodrug(s) can be conveniently delivered in the form of an aerosol spray from pressurized packs or a nebulizer with the use of a suitable propellant (e.g., dichlorodifluoromethane, trichlorofluoromethane, dichlorotetrafluoroethane, fluorocarbons, carbon dioxide, or other suitable gas). In the case of a pressurized aerosol, the dosage unit can be determined by providing a valve to deliver a metered amount. Capsules and cartridges for use in an inhaler or insufflator (for example, capsules and cartridges comprised of gelatin) can be formulated containing a powder mix of the compound and a suitable powder base such as lactose or starch.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution, and isotonic sodium chloride solution. The compounds may also be administered in the form of suppositories for rectal or urethral administration of the drug.

For topical use, creams, ointments, jellies, gels, solutions, suspensions, etc., containing the compounds described herein, can be employed, in some embodiments, the compounds described herein can be formulated for topical administration with polyethylene glycol (PEG). These formulations may optionally comprise additional pharmaceutically acceptable ingredients such as diluents, stabilizers, and/or adjuvants.

Included among the devices which can be used to administer compounds described herein, are those well-known in the art, such as metered dose inhalers, liquid nebulizers, dry powder inhalers, sprayers, thermal vaporizers, and the like. Other suitable technology for administration of particular compounds of the invention, includes electrohydrodynamic aerosolizers. As those skilled in the art will recognize, the formulation of compounds, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of compounds in the aerosol. For example, shorter periods of administration can be used at higher concentrations of compounds in the nebulizer solution. Devices such as metered dose inhalers can produce higher aerosol concentrations and can be operated for shorter periods to deliver the desired amount of compounds in some embodiments. Devices such as dry powder inhalers deliver active agent until a given charge of agent is expelled from the device, in this type of inhaler, the amount of compounds in a given quantity of the powder determines the dose delivered in a single administration.

Formulations of compounds described herein for administration from a dry powder inhaler may typically include a finely divided dry powder containing compounds, but the powder can also include a bulking agent, buffer, carrier, excipient, another additive, or the like. Additives can be included in a dry powder formulation of compounds of the invention, for example, to dilute the powder as required for delivery from the particular powder inhaler, to facilitate processing of the formulation, to provide advantageous powder properties to the formulation, to facilitate dispersion of the powder from the inhalation device, to stabilize to the formulation e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; and the like.

For prolonged delivery, the compound(s) or prodrug(s) described herein can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives (e.g., as a sparingly soluble salt). Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in, for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems can be employed. Liposomes and emulsions are weft-known examples of delivery vehicles that can be used to deliver active compound(s) or prodrug(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device can be accompanied by instructions for administration.

The compound(s) or prodrug(s) described herein, or compositions thereof, will generally be used in an amount effective to achieve the intended result, for example, in an amount effective to treat or prevent the particular condition being treated. The compound(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a compound to a patient suffering from an diarrhea provides therapeutic benefit not only when the diarrhea is eradicated or ameliorated, but also when the patient reports a decrease in the severity or duration of the symptoms associated with the diarrhea. Therapeutic benefit also includes halting or slowing the progression of the disease, regardless of whether improvement is realized.

The amount of compound administered will depend upon a variety of factors, including, for example, the particular condition being treated, the mode of administration, the severity of the condition being treated, the age and weight of the patient, the bioavailability of the particular active compound. Determination of an effective dosage is well within the capabilities of those skilled in the art. As known by those of skill in the art, the preferred dosage of compounds of the invention will also depend on the age, weight, general health, and severity of the condition of the individual being treated. Dosage may also need to be tailored to the sex of the individual and/or the lung capacity of the individual, where administered by inhalation. Dosage, and frequency of administration of the compounds or prodrugs thereof, will also depend on whether the compounds are formulated for treatment of acute episodes of a condition or for the prophylactic treatment of a disorder. A skilled practitioner will be able to determine the optimal dose for a particular individual.

For prophylactic administration, the compound can be administered to a patient at risk of developing one of the previously described conditions. For example, if it is unknown whether a patient is allergic to a particular drug, the compound can be administered prior to administration of the drug to avoid or ameliorate an allergic response to the drug. Alternatively, prophylactic administration can be applied to avoid the onset of symptoms in a patient diagnosed with the underlying disorder.

Effective dosages can be estimated initially from in vitro assays. For example, an initial dosage for use in animals can be formulated to achieve a circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of skilled artisans. For guidance, the reader is referred to Firgl & Woodbury, “General Principles,” GOODMAN AND GILMAN'S THE PHARMACEUTICAL BASIS OF THERAPEUTICS, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

The foregoing disclosure pertaining to the dosage requirements for the compounds of the invention is pertinent to dosages required for prodrugs, with the realization, apparent to the skilled artisan, that the amount of prodrug(s) administered will also depend upon a variety of factors, including, for example, the bioavailability of the particular prodrug(s) and the conversation rate and efficiency into active drug compound under the selected route of administration. Determination of an effective dosage of prodrug(s) for a particular use and mode of administration is well within the capabilities of those skilled in the art.

Also provided are kits for administration of the compounds described herein, prodrug thereof, or pharmaceutical formulations comprising the compound that may include a dosage amount of at least one compound or a composition comprising at least one compound, as disclosed herein. Kits may further comprise suitable packaging and/or instructions for use of the compound. Kits may also comprise a means for the delivery of the at least one compound or compositions comprising at least one compound of the invention, such as an inhaler, spray dispenser (e.g., nasal spray), syringe for injection, or pressure pack for capsules, tables, suppositories, or other device as described herein.

Other types of kits provide the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch, or inhalant.

The kits may include other therapeutic compounds for use in conjunction with the compounds described herein. These compounds can be provided in a separate form or mixed with the compounds of the present invention. The kits will include appropriate instructions for preparation and administration of the composition, side effects of the compositions, and any other relevant information. The instructions can be in any suitable format, including, but not limited to, printed matter, videotape, computer readable disk, or optical disc.

In one embodiment, this invention provides a kit comprising a compound selected from the compounds described herein or a prodrug thereof, packaging, and instructions for use.

In another embodiment, this invention provides a kit comprising the pharmaceutical formulation comprising a compound selected from the compounds described herein or a prodrug thereof and at least one pharmaceutically acceptable excipient, diluent, preservative, stabilizer, or mixture thereof, packaging, and instructions for use. In another embodiment, kits for treating an individual who suffers from or is susceptible to the conditions described herein are provided, comprising a container comprising a dosage amount of a compound of this invention or composition, as disclosed herein, and instructions for use. The container can be any of those known in the art and appropriate for storage and delivery of oral, intravenous, topical, rectal, urethral, or inhaled formulations.

Kits may also be provided that contain sufficient dosages of the compounds or composition to provide effective treatment for an individual for an extended period, such as a week, 2 weeks, 3, weeks, 4 weeks, 6 weeks, or 8 weeks or more.

### E. General Synthesis of the Compounds of the Invention

The compounds and prodrugs described herein can be synthesized via a variety of different synthetic routes using commercially available starting materials and/or starting materials prepared by conventional synthetic methods. It will also be appreciated by those skilled in the art that in the process described below, the functional groups of intermediate compounds may need to be protected by suitable protecting groups.

The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein. Examples of functional groups include hydroxy, amino, mercapto and carboxylic acid.

Thus, “protecting group” refers to a group of atoms that, when attached to a reactive functional group in a molecule, mask, reduce or prevent the reactivity of the functional group. Typically, a protecting group can be selectively removed as desired during the course of a synthesis. Examples of protecting groups can be found in Greene and Wuts, as mentioned above, and, additionally, in Harrison et al., COMPENDIUM OF SYNTHETIC ORGANIC METHODS, Vols, 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”), and the like. Representative hydroxyl protecting groups include, but are not limited to, those where the hydroxyl group is either acylated to form acetate and benzoate esters or alkylated to harm benzyl and trityl ethers, as well as alkyl ethers, tetrahydropyranyl ethers, trialkylsilyl ethers (e.g. TMS or TIPPS groups), aryl silyl ethers (e.g., triphenylsilyl ether), mixed alkyl and aryl substituted silyl ethers, and allyl ethers.

The following reaction Schemes illustrate methods to make compounds described herein. It is understood that one of ordinary skill in the art would be able to make the compounds by similar methods or by methods known to one skilled in the art. In general, starting components may be obtained from sources such as Aldrich, or synthesized according to sources known to those of ordinary skill in the art (see, e.g., Smith and March, MARCH'S ADVANCED ORGANIC CHEMISTRY: REACTIONS, MECHANISMS, AND STRUCTURE, 5th edition (Wiley Interscience, New York)). Moreover, the various substituted groups (e.g., R¹, R², R³, R⁴, R⁵, R⁶, p etc.) of the compounds of the invention may be attached to the starting components, intermediate components, and/or final products according to methods known to those of ordinary skill in the art.

A variety of exemplary synthetic routes that can be used to synthesize the compounds are described in Scheme I below. Specifically, compounds of formula I can be synthesized using the methods disclosed hereinbelow. These methods can be routinely adapted to synthesize the compounds and prodrugs described herein.

In one exemplary embodiment, various compounds of formula I can be synthesized from esters I-A as illustrated in Scheme I, below:

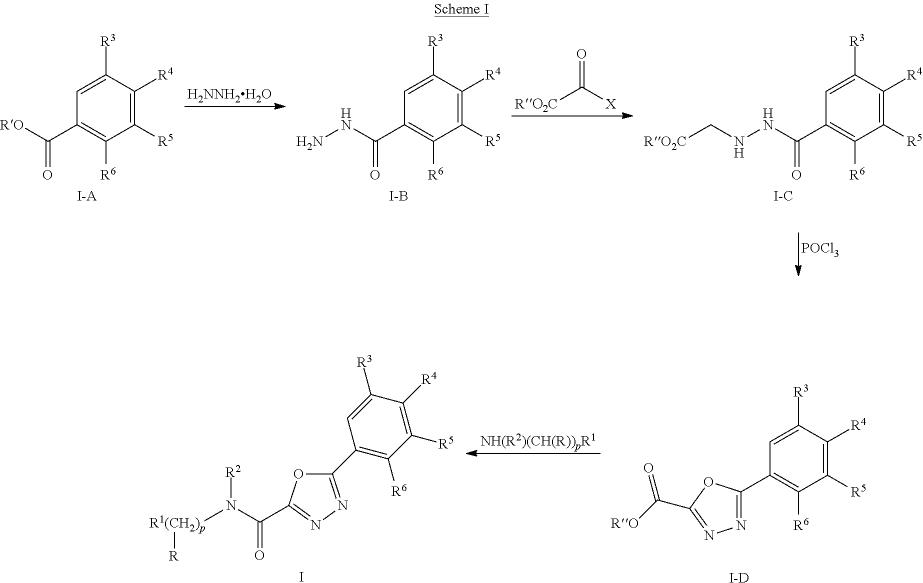

In Scheme I, the groups R, R¹, R², R³, R⁴, R⁵, p, and R⁶ are as defined herein, X is halo, and R′ and R″ are independently lower alkyl. The starting esters I-A can be purchased from commercial sources or prepared using standard techniques of organic chemistry. Typically, ester I-A is reacted with hydrazine hydrate to give hydrazide I-B under standard conditions. Hydrazide I-B is then converted to compound I-C by reacting with a halooxoacetate. Compound I-C is then cyclized to 1,3,4-oxadiazole-2-carboxylate I-D via treatment with POCl₃. The 1,3,4-oxadiazole-2-carboxylate I-D is then reacted with suitable amines to give compounds of formula I. In each of the above recited steps, the product may be recovered by conventional methods such as evaporation, chromatography, precipitation, crystallization, and the like or, alternatively, used in the next step without purification and/or isolation. The reactions depicted in Scheme I may proceed more quickly when the reaction solutions are rapidly heated by, e.g., a microwave.

Compounds I-A can be purchased from commercial sources or prepared using standard techniques of organic chemistry. For example, when R³, R⁴, and R⁶ are each hydrogen and R⁵ is aminosulfonyl, or when R³, R⁵, and R⁶ are each hydrogen and R⁴ is aminosulfonyl, ester I-A can be synthesized in one step via sulfonylation of the corresponding amine (ester I-A wherein R⁴ or R⁵ is NH₂) using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compound I-A may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons. Inc., New York (1999) and the references cited therein (hereinafter “Greene & Wuts”).

The following examples are intended to illustrate the various embodiments of this invention.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

In the examples below as well as throughout the application, the following abbreviations have the following meanings, if not defined, the terms have their generally accepted meanings.
APCI=atmospheric pressure chemical ionization
ATP=adenosine tri-phospate
br=broad
BuOH=butanol
d=doublet
(CF₃SO₂)₂)=trifluoromethanesulfonic anhydride
CH₂Cl₂=dichloromethane
CH₃CN=acetonitrile
DMEM=Dulbecco's modified eagle's medium
DMSO=dimethylsulfoxide
EGTA=ethylene glycol tetraacetic acid
Et₃N=triethylamine
EtOH=ethanol
EtOAc=ethyl acetate
FBS=fetal bovine serum
g=gram
h=hour
LC=liquid chromatography
LCMS=liquid chromatography mass spectrometry
m=multiplet
m/z=mass/Charge
Me=methyl
MeOH=methanol
mg=milligram
MgSO₄=magnesium sulfate
MHz=megahertz
min=minute
mL=milliliter
mm=millimeter
mM=milimolar
mmol=millimole
ms=millisecond
MS=mass spectrum
mV=millivolt
MΩ=megaohm
N=normal
NH₂NH₂.H₂O=hydrazine hydrate
nM=nanomolar
nm=nanometer
NMR=nuclear magnetic resonance
POCl₃=phosphorus oxychloride
ppm=parts per million
q=quartet
rt=room temperature
Rt=retention time
s=singlet
SSC=standard saline citrate
t=triplet
TEA=triethylamine
THF=tetrahydrofuran
UV=ultraviolet
v/v=volume/volume
μg=microgram
μL=microliter
μm=micrometer
μM=micromolar

### General Synthetic Methods

Unless otherwise stated, all chemicals were purchased from commercial suppliers and used without further purification. NMR spectra were recorded on Bruker 400 MHz, spectrometers. Chemical shifts are reported in parts per million downfield from the internal standard Me₄Si (0.0 ppm) for CDCl₃ solutions. For DMSO-d₆ solutions, calibration was done on the solvent peak at 2.49 ppm.

### Standard Acidic LC-MS Conditions

### (10 cm_esci_formic or 10 cm_apci_formic)

A Phenomenex Luna 5 μm C18 (2), 100×4.6 mm (plus guard cartridge) column using an acetonitrile (far UV grade) with 0.1% (v/v) formic acid:Water (high purity via Elga UHQ unit) with 0.1% formic acid gradient was used. The flow rate was 2 mL/min. UV detection was done using a Waters diode array detector (start range 210 nm, end range 40 nm, range interval 4.0 nm). Mass detection was via a single quadrapole LCMS instrument. Ionization is either ESCi™ or APCI dependent on compound types. The gradient used ran from 95% of aqueous solvent at time 0.00 min to 5% of aqueous solvent at 3.50 min. This percentage was then held for a further 2 min.

### Standard Bask LC-MS Conditions

### (10 cm_esci_bicarb or 10 cm_apci_bicarb)

A Waters Xterra MS 5 μm C18, 100×4.6 mm (Plus guard cartridge) column using an acetonitrile (far UV grade): water (high purity via Elga UHQ unit) with 10 mM ammonium bicarbonate (ammonium hydrogen carbonate) gradient was used. The flow rate was 2 mL/min. UV detection was done using a Waters diode array detector (start range 210 nm, end range 400 nm, range interval 4.0 nm). Mass detection was via a single quadrapole LCMS instrument. Ionization is either ESCi™ or APCI dependent on compound types. The gradient used ran from 95% of aqueous solvent at time 0.00 min to 5% of aqueous solvent at 3.50 min. This percentage was then held for a further 2 min.

### Example 1

### Preparation of 5-(3,5-Dichloro-4-hydroxyphenyl)-N-(4-phenoxybenzyl)-1,3,4-oxadiazole-2-carboxamide (Compound 2)

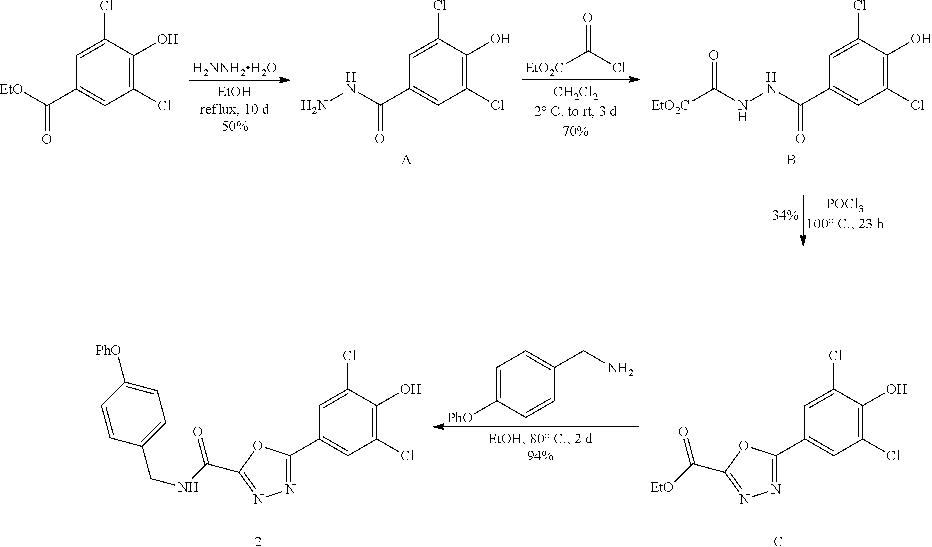

### Step 1: 3,5-Dichloro-4-hydroxybenzohydrazide (Compound A)

To a mixture of ethyl 3,5-dichloro-4-hydroxybenzoate (23.5 g, 100 mmol) in ethanol (250 mL) was added hydrazine monohydrate (6 mL, 130 mmol) and the mixture was heated at reflux for 18 h. More hydrazine monohydrate (18 mL, 389 mmol) was added and the mixture was heated at reflux for another 9 d. The mixture was cooled to room temperature and the resulting solid was collected by filtration, washed with ethanol and dried to leave 11.02 g (50%) of the title compound as a white solid. ¹H NMR δ (ppm) (DMSO-d₆): 7.63 (2H, s), 9.19 (1H, s).

### Step 2: Ethyl 2-(2-(3,5-dichloro-4-hydroxybenzoyl)hydrazinyl)-2-oxoacetate (Compound B)

To a stirred mixture of 3,5-dichloro-4-hydroxybenzohydrazide (2.00 g, 9.05 mmol) in anhydrous dichloromethane (50 mL) under nitrogen, cooled in an ice-water bath at 2° C., was added ethyl chlorooxoacetate (1.52 mL, 13.6 mmol) dropwise. After 20 min, the cooling bath was removed and stirring was continued for 3 d. The mixture was filtered and the solid was washed with dichloromethane twice and dried at 60° C. under vacuum to give 2.046 g (70%) of the title compound as a white solid. ¹H NMR δ (ppm) (DMSO-d₆): 1.27-1.37 (3H, m), 4.27-4.36 (2H, m), 7.94 (2H, s), 10.66 (1H, s), 10.97 (1H, s).

### Step 3: Ethyl 5-(3,5-dichloro-4-hydroxyphenyl)-1,3,4-oxadiazole-2-carboxylate (Compound C)

A mixture of ethyl 2-(2-(3,5-dichloro-4-hydroxybenzoyl)hydrazinyl)-2-oxoacetate (2.05 g, 6.38 mmol) in phosphorous oxychloride (60 mL) was stirred at 100° C. for 23 h. The excess POCl₃ was evaporated and the residue was partitioned between water (50 mL) and dichloromethane (50 mL). The aqueous layer was extracted further with dichloromethane (2×50 mL) and the combined organic extracts were washed with brine (30 mL), dried (MgSO₄) and evaporated. The residue was preabsorbed onto silica gel and purified by flash chromatography (silica gel, 2% MeOH/CH₂Cl₂) to give 0.6649 g (34%) of the title compound as a white solid, ¹H NMR δ (ppm) (DMSO-d₆): 1.40 (3H, t, J=7.11 Hz), 4.49 (2H, q, J=7.11 Hz), 8.03 (2H, s).

### Step 4: 5-(3,5-Dichloro-4-hydroxyphenyl)-N-(4-phenoxybenzyl)-1,3,4-oxadiazole-2-carboxamide (Compound 2)

A mixture of ethyl 5-(3,5-dichloro-4-hydroxyphenyl)-1,3,4-oxadiazole-2-carboxylate (0.3003 g, 0.990 mmol) and 4-phenoxybenzylamine (0.5922 g, 2.97 mmol) in ethanol (10 mL) was stirred at 80° C. under nitrogen for 2 d. The mixture was partitioned between dilute aqueous HCl (50 mL) and ethyl acetate (75 mL). The aqueous layer was extracted further with ethyl acetate (50 mL) and the combined extracts were washed with brine (30 mL), dried (MgSO₄) and evaporated. The residue was purified by flash chromatography (silica gel, 2% MeOH/CH₂Cl₂) to give 0.4235 g (94%) of the title compound as a white solid. ¹H NMR δ (ppm) (DMSO-d₆): 4.53 (2H, d, J=6.12 Hz), 7.00-7.05 (4H, m), 7.16 (1H, t, J=7.40 Hz), 7.39-7.44 (4H, m), 8.07 (2H, s), 9.90 (1H, t, J=6.15 Hz); LCMS (10 cm_ESI_bicarb) t_{R} 2.58 min; m/z 456/458/460 [M+H]⁺.

Following the procedure set forth above, but employing a suitable amine in Step 4, the following compounds were prepared:

### 5-(3,5-dichloro-4-hydroxyphenyl)-N-(3-(trifluoromethoxy)benzyl)-1,3,4-oxadiazole-2-carboxamide, ammonium salt (Compound 1)

LCMS (10 cm_ESI_bicarb) Rt 2.48 min; m/z 446/448/450 [M+H]−; ¹H NMR δ (ppm) (DMSO-d₆): 4.56 (2H, d, J=6.18 Hz), 7.15 (4H, s), 7.30 (1H, d, J=8.24 Hz), 7.38 (1H, s), 7.42 (1H, d, J=7.81 Hz), 7.52 (1H, t, J=7.89 Hz), 7.70 (2H, s), 9.77 (1H, t, J=6.22 Hz).

### (4-benzylpiperidin-1-yl)(5-(3,5-dichloro-4-hydroxyphenyl)-1,3,4-oxadiazol-2-yl)methanone (Compound 3)

LCMS (10 cm_ESI_formic) Rt 3.97 min; m/z 432/434/436 [M+H]+; ¹H NMR δ (ppm) (DMSO-d₆): 1.21-1.30 (2H, m), 1.67-1.79 (2H, m), 1.90-1.92 (1H, m), 2.59 (2H, d, J=7.17 Hz), 2.90 (1H, d, J=3.06 Hz), 3.23 (1H, s), 4.46 (2H, m), 7.20-7.25 (3H, m), 7.33 (2H, t, J=7.39 Hz), 7.99 (2H, s), 11.38 (1H, s).

### (5-(3,5-dichloro-4-hydroxyphenyl)-1,3,4-oxadiazol-2-yl)(4-(3-(trifluoromethyl)phenyl)piperazin-1-yl)methanone (Compound 4)

LCMS (10 cm_ESI_formic) Rt 4.41 min; m/z 487/489/491 [M+H]+; ¹H NMR δ (ppm) (DMSO-d₆): 3.43 (4H, s), 3.89 (2H, t, J=4.85 Hz), 4.16 (2H, t, J=4.71 Hz), 7.15 (1H, d, J=7.60 Hz), 7.27 (1H, s), 7.31 (1H, d, J=8.71 Hz), 7.49 (1H, t, J=7.95 Hz), 8.02 (2H, s), 11.41 (1H, s).

### N-(4-tert-butylbenzyl)-5-(3,5-dichloro-4-hydroxyphenyl)-1,3,4-oxadiazole-2-carboxamide (Compound 5)

LCMS (10 cm_ESI_bicarb) Rt 3.82 min; m/z 420/422/424 [M+H]+; ¹H NMR δ (ppm) (DMSO-d₆): 1.30 (9H, s), 4.50 (2H, d, J=6.10 Hz), 7.32 (2H, d, J=8.14 Hz), 7.40 (2H, d, J=8.20 Hz), 8.06 (2H, s), 9.86 (1H, t, J=6.12 Hz), 11.38 (1H, s).

### N-benzhydryl-5-(3,5-dichloro-4-hydroxyphenyl)-1,3,4-oxadiazole-2-carboxamide (Compound 6)

LCMS (10 cm_ESI_formic) Rt 4.34 min; m/z 438/440/442 [M−H]−; ¹H NMR δ (ppm) (DMSO-d₆): 6.46 (1H, d, J=8.73 Hz), 7.32-7.37 (2H, m), 7.38-7.46 (8H, m), 8.08 (2H, s), 10.24 (1H, d, J=8.80 Hz), 11.39 (1H, s).

### 5-(3,5-dichloro-4-hydroxyphenyl)-N-(3,4-dichlorobenzyl)-N-methyl-1,3,4-oxadiazole-2-carboxamide (compound 12)

LCMS (10 cm_ESI_Formic) Rt 3.92 min; m/z 444/446/448 [M−H]−; ¹H NMR δ (ppm) (DMSO-d₆): 3.03 and 3.39 (3H, s), 4.78 and 5.04 (2H, s), 7.41 (1H, td, J=8.28, 2.07 Hz), 7.66-7.72 (2H, m), 7.90 (1H, s), 8.02 (1H, s). Rotameric effect observed in the NMR.

### 5-(3,5-dichloro-4-hydroxyphenyl)-N-methyl-N-(3-phenoxybenzyl)-1,3,4-oxadiazole-2-carboxamide (Compound 13)

LCMS (10 cm_ESI_Formic) Rt 3.97 min; m/z 444/446/448 [M−H]−; ¹H NMR δ (ppm) (DMSO-d₆): 3.03 and 3.32 (3H, s), 4.77 and 5.02 (2H, s), 6.97 (1H, m), 7.02-7.07 (3H, m), 7.14-7.21 (2H, m), 7.38-7.47 (3H, m), 7.93 (1H, s), 8.03 (1H, s). Rotamers.

### 5-(3,5-dichloro-4-hydroxyphenyl)-N-(3-phenoxybenzyl)-1,3,4-oxadiazole-2-carboxamide (Compound 14)

LCMS (10 cm_ESI_Formic) Rt 3.77 min; m/z 456/458 [M+H]−; ¹H NMR δ (ppm) (DMSO-d₆): 4.53 (2H, d, J=6.17 Hz), 6.92 (1H, dd, J=8.16, 2.48 Hz), 7.00-7.10 (3H, m), 7.16 (2H, m), 7.36-7.46 (3H, m), 8.06 (1H, s), 9.90 (1H, t, J=6.18 Hz).

### 5-(3,5-dichloro-4-hydroxyphenyl)-N-(2,2-diphenylethyl)-1,3,4-oxadiazole-2-carboxamide (Compound 15)

LCMS (10 cm_ESI_Formic) Rt 3.79 min; m/z 452/454 [M+H]−; ¹H NMR δ (ppm) (DMSO-d₆): 4.00 (2H, t, J=6.76 Hz), 4.49 (1H, t, J=7.89 Hz), 7.19-7.26 (2H, m), 7.30-7.40 (8H, m), 8.01 (2H, s), 9.43 (1H, t, J=5.68 Hz).

### N-(3-(benzyloxy)benzyl)-5-(3,5-dichloro-4-hydroxyphenyl)-1,3,4-oxadiazole-2-carboxamide (Compound 16)

LCMS (10 cm_ESI_Formic) Rt 3.76 min; m/z 470/472 [M+H]−; ¹H NMR δ (ppm) (DMSO-d₆): 4.52 (2H, d, J=6.17 Hz), 5.12 (2H, s), 6.93-6.99 (2H, m), 7.04 (1H, s), 7.27-7.37 (2H, m), 7.40 (2H, t, J=7.39 Hz), 7.47 (2H, d, J=7.49 Hz), 8.07 (2H, s). 9.87 (1H, t, J=6.14 Hz).

### N-(4-(benzyloxy)benzyl)-5-(3,5-dichloro-4-hydroxyphenyl)-1,3,4-oxadiazole-2-carboxamide (Compound 18)

LCMS (10 cm_ESI_Formic) Rt 3.77 min; m/z 468/470 [M−H]−; ¹H NMR δ (ppm) (DMSO-d₆): 4.46 (2H, d, J=6.11 Hz), 5.13 (2H, s), 7.02 (2H, d, J=8.35 Hz), 7.28-7.50 (7H, m), 8.07 (2H, s), 9.83 (1H, t, J=6.12 Hz).

### N-(biphenyl-3-ylmethyl)-5-(3,5-dichloro-4-hydroxyphenyl)-1,3,4-oxadiazole-2-carboxamide (Compound 19)

LCMS (10 cm_ESI_Bicarb) Rt 2.48 min; m/z 438/440/441 [M−H]−; ¹H NMR δ (ppm) (DMSO-d₆): 4.62 (2H, d, J=6.13 Hz), 7.37-7.43 (2H, m), 7.46-7.54 (3H, m), 7.60 (1H, d, J=7.76 Hz), 7.69 (3H, t, J=3.74 Hz), 8.02 (2H, s), 9.92 (1H, t, J=6.13 Hz).

### 5-(3,5-dichloro-4-hydroxyphenyl)-N-(3-fluoro-5-(trifluoromethyl)benzyl)-1,3,4-oxadiazole-2-carboxamide (Compound 21)

LCMS (10 cm_ESI_Formic) Rt 3.64 min; m/z 448/450/451 [M−H]−; ¹H NMR δ (ppm) (DMSO-d₆): 4.65 (2H, d, J=6.10 Hz), 7.54-7.67 (3H, m), 8.06 (2H, s), 9.97 (1H, t, J=6.12 Hz), 11.41 (1H, br s).

### 5-(3,5-dichloro-4-hydroxyphenyl)-N-(4-(trifluoromethoxy)benzyl)-1,3,4-oxadiazole-2-carboxamide (Compound 22)

LCMS (10 cm_ESI_Bicarb) Rt 2.36 min; m/z 448/450 [M−H]−; ¹H NMR δ (ppm) (DMSO-d₆): 4.57 (2H, d, J=6.12 Hz), 7.39 (2H, d, J=8.20 Hz), 7.53 (2H, d, J=8.39 Hz), 8.07 (2H, s), 9.94 (1H, t, J=6.13 Hz).

### 5-(3,5-dichloro-4-hydroxyphenyl)-N-(4-fluoro-3-(trifluoromethyl)benzyl)-1,3,4-oxadiazole-2-carboxamide (Compound 24)

LCMS (10 cm_ESI_Formic) Rt 3.61 min; m/z 450/452/454 [M+H]−; ¹H NMR δ (ppm) (DMSO-d₆): 4.60 (2H, d, J=6.11 Hz), 7.54 (1H, t, J=9.75 Hz), 7.74-7.84 (2H, m), 8.04 (2H, s), 9.94 (1H, t, J=6.13 Hz).

### N-(1-(4-chlorophenyl)ethyl)-5-(3,5-dichloro-4-hydroxyphenyl)-1,3,4-oxadiazole-2-carboxamide (Compound 26)

LCMS (10 cm_ESI_Bicarb) Rt 2.29 min; m/z 412/414 [M+H]−; ¹H NMR δ (ppm) (DMSO-d₆): 1.56 (3H, d, J=7.02 Hz), 5.22 (1H, t, J=7.41 Hz), 7.41-7.51 (4H, m), 8.07 (2H, s). 9.81 (1H, d, J=8.10 Hz).

### Example 2

### Preparation of N-(4-phenoxybenzyl)-5-(3-(trifluoromethylsulfonamido)phenyl)-1,3,4-oxadiazole-2-carboxamide (Compound 7)

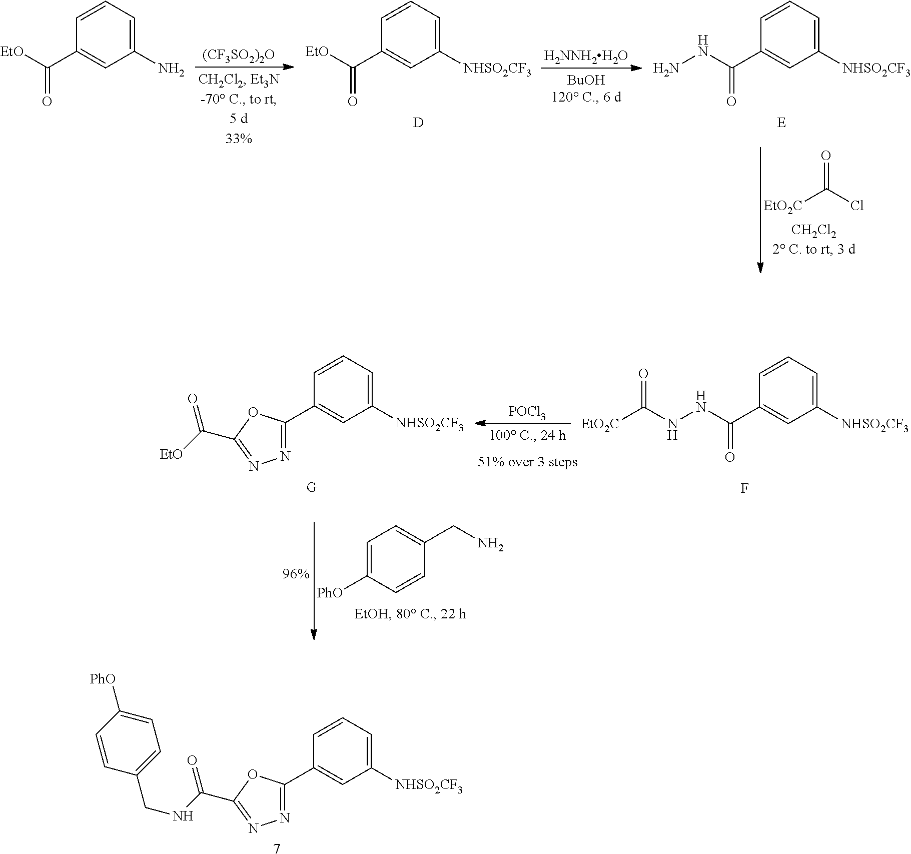

### Step 1: Ethyl 3-(trifluoromethylsulfonamido)benzoate (Compound D)

To a stirred solution of ethyl 3-aminobenzoate (3.32 g, 20.1 mmol) in anhydrous dichloromethane under nitrogen cooled in an ice-water bath at 3° C. was added, dropwise over 5 min, trifluoromethanesulfonic anhydride (4.06 mL, 24.1 mmol). After 80 min the cooling bath was removed and the mixture was stirred under nitrogen for 26 h. The mixture was recooled with ice-water bath and more trifluoromethanesulfonic anhydride (4.06 mL, 24.1 mmol) was added and the mixture was stirred at room temperature for another 16 h. The mixture was then cooled to −70° C. and triethylamine (6.72 mL, 48.2 mmol) was added dropwise over 20 min, keeping the temperature <−55° C. After the addition was complete the resulting solution was stirred at room temperature for 3 d. The reaction mixture was diluted with dichloromethane (50 mL) and washed with 1 N hydrochloric acid (100 mL), then brine (50 mL). The remaining organic layer was dried (MgSO₄) and evaporated. The residue was purified by flash chromatography (silica gel, 15% EtOAc/petroleum ether) to afford 1.97 g (33%) of the title compound as a white solid: ¹H NMR δ (ppm) (CDCl₃): 1.41 (3H, t, J=7.13 Hz), 4.42 (2H, q, J=7.13 Hz), 7.23 (1H, s), 7.49 (1H, t, J=7.86 Hz), 7.56-7.60 (1H, m), 7.96-8.00 (2H, m).

### Step 2: 1,1,1-Trifluoro-N-(3-(hydrazinecarbonyl)phenyl)methanesulfonamide (Compound E)

To a mixture of ethyl 3-(trifluoromethylsulfonamido)benzoate (1.95 g, 6.56 mmol) in 1-butanol (10 mL) was added hydrazine monohydrate (0.759 mL, 16.4 mmol) and the mixture was heated at 120° C. for 5 d. More hydrazine monohydrate (0.304 mL, 6.56 mmol) was added and the mixture was heated at reflux for another 1 d. The solvent was evaporated and the oily residue was azeotroped with ethanol to leave 2.34 g of the crude title compound as an oil.

### Step 3: Ethyl 2-oxo-2-(2-(3-(trifluoromethylsulfonamido)benzoyl)hydrazinyl)acetate (Compound F)

To a stirred mixture of crude 1,1,1-trifluoro-N-(3-(hydrazinecarbonyl)phenyl)methanesulfonamide (2.34 g) in anhydrous dichloromethane (35 mL) under nitrogen, cooled to 2° C., was added ethyl chlorooxoacetate (1.10 mL, 9.85 mmol) dropwise. After 10 min, the cooling bath was removed and stirring was continued for 3 d. The mixture was recooled to 3° C., and more ethyl chlorooxoacetate (0.37 mL, 3.31 mmol) was added dropwise. The mixture was stirred at room temperature under nitrogen for another 3 h. The mixture was filtered and the solid was washed with dichloromethane twice and dried at 60° C. under vacuum to give 2.8773 g of the crude title compound as a white solid, ¹H NMR δ (ppm) (DMSO-d₆): 1.34 (3H, t, J=7.13 Hz), 4.34 (2H, q, J=7.19 Hz), 7.54 (2H, d, J=8.67 Hz), 7.61 (1H, t, J=7.84 Hz), 7.80 (1H, t, J=1.93 Hz), 7.84 (1H, d, J=7.85 Hz), 10.78 (1H, s), 11.00 (1H, s).

### Step 4: Ethyl 5-(3-(trifluoromethylsulfonamido)phenyl-1,3,4-oxadiazole-2-carboxylate (Compound G)

A mixture of crude ethyl 2-oxo-2-(2-(3-(trifluoromethylsulfonamido)benzoyl)hydrazinyl)acetate (1.00 g) in phosphorous oxychloride (30 mL) was stirred at 100° C. for 24 h. The excess POCl₃ was evaporated and the residue was partitioned between water (30 mL) and dichloromethane (50 mL), using a little methanol (3 mL) to dissolve the remaining solid in the flask to transfer to a separating funnel. The aqueous layer was extracted further with dichloromethane (2×30 mL) and the combined organic extracts were washed with brine (30 mL), dried (MgSO₄) and evaporated. The residue was preabsorbed onto silica gel and purified by flash chromatography (silica gel, 2% MeOH/CH₂Cl₂) to give 0.42 g (51% over 3 steps) of the title compound as a pale pink solid. ¹H NMR δ (ppm) (DMSO-d₆): 1.41 (3H, t, J=7.11 Hz), 4.50 (2H, q, J=7.11 Hz), 7.59 (1H, d, J=8.31 Hz), 7.71 (1H, t, J=7.93 Hz), 7.93-7.99 (2H, m).

### Step 5: N-(4-Phenoxybenzyl)-5-(3-(trifluoromethylsulfonamido)phenyl)-1,3,4-oxadiazole-2-carboxamide (Compound 7)

A mixture of ethyl 5-(3-(trifluoromethylsulfonamido)phenyl)-1,3,4-oxadiazole-2-carboxylate (0.2003 g, 0.548 mmol) and 4-phenoxybenzylamine (0.3278 g, 1.65 mmol) in ethanol (7 mL) was stirred at 80° C. under nitrogen for 22 h. The mixture was partitioned between dilute aqueous HCl (60 mL) and ethyl acetate (40 mL). The aqueous layer was extracted further with ethyl acetate (40 mL) and the combined extracts were washed with brine (15 mL), dried (MgSO4) and evaporated. The residue was purified by flash chromatography (silica gel, 2% MeOH/CH₂Cl₂) to give 0.2739 g (96%) of the title compound as a white solid. ¹H NMR δ (ppm) (DMSO-d₆): 4.52 (2H, d, J=5.97 Hz), 7.03 (4H, d, J=7.91 Hz), 7.16 (1H, t, J=7.35 Hz), 7.39-7.45 (4H, m), 7.54 (1H, d, J=8.00 Hz), 7.68 (1H, t, J=7.90 Hz), 7.95 (2H, m), 9.95 (1H, t, J=6.01 Hz); LCMS (10 cm_ESI_Formic_) t_{R} 4.86 min; m/z 519 [M+H]⁺.

Following the procedure set forth above, but employing a suitable amine in Step 5, the following compounds were prepared:

### N-(3-(5-(4-benzylpiperidine-1-carbonyl)-1,3,4-oxadiazol-2-yl)phenyl)-1,1,1-trifluoromethanesulfonamide (Compound 8)

LCMS (10 cm_ESI_Formic_MeOH) Rt 4.52 min; m/z 495 [M+H]⁺; ¹H NMR δ (ppm) (DMSO-d₆): 1.18-1.33 (2H, m), 1.66-1.80 (2H, m), 1.92 (1H, m), 2.60 (2H, d, J=7.60 Hz), 2.82-2.94 and 3.19-3.30 (2H, m), 4.43-4.55 (2H, m), 7.18-7.25 (3H, m), 7.29-7.36 (2H, m), 7.43 (1H, d, J=7.39 Hz), 7.56 (1H, t, J=7.40 Hz), 7.75 (1H, d, J=7.76 Hz), 7.85 (1H, s).

### N-(4-tert-butylbenzyl)-5-(3-(trifluoromethylsulfonamido)phenyl)-1,3,4-oxadiazole-2-carboxamide (Compound 9)

LCMS (10 cm_ESI_Bicarb_CH₃CN) Rt 3.05 min; m/z 483 [M+H]+; ¹H NMR δ (ppm) (DMSO-d₆): 1.30 (9H, s), 4.49 (2H, d, J=6.14 Hz), 7.32 (2H, d, J=8.08 Hz), 7.40 (2H, d, J=8.13 Hz), 7.54 (1H, d, J=8.01 Hz), 7.67 (1H, t, J=7.96 Hz), 7.90-7.95 (2H, m), 9.91 (1H, t, J=6.24 Hz).

### N-(3,4-dichlor benzyl)-N-methyl-5-(3-(trifluoromethylsulfonamido)phenyl)-1,3,4-oxadiazole-2-carboxamide (Compound 10)

LCMS (10 cm_ESI_Bicarb_CH₃CN) Rt 2.99 min; m/z 509/511/513 [M+H]+; ¹H NMR δ (ppm) (DMSO-d₆): 3.02 and 3.41 (3H, two s), 4.79 and 5.08 (2H, two s), 7.41 and 7.44 (1H, two dd, J=8.30, 2.16 and 8.32, 2.17 Hz), 7.54 (1H, two s), 7.65-7.74 (3H, m), 7.93 (2H, m).

### 1,1,1-trifluoro-N-(3-(5-(4-(3-(trifluoromethyl)phenyl)piperazine-1-carbonyl)-1,3,4-oxadiazol-2-yl)phenyl)methanesulfonamide (Compound 11)

LCMS (10 cm_ESI_Bicarb_CH₃CN) Rt 3.06 min; m/z 550 [M+H]+; ¹H NMR δ (ppm) (DMSO-d₆): 3.41-3.50 (4H, m), 3.86-3.91 (2H, m), 4.10-4.28 (2H, m), 7.15 (1H, d, J=7.62 Hz), 7.28 (1H, s), 7.31 (1H, d, J=8.92 Hz), 7.44 (1H, d, J=8.24 Hz), 7.49 (1H, t, J=8.00 Hz), 7.57 (1H, t, J=7.62 Hz), 7.78 (1H, d, J=7.81 Hz), 7.88 (1H, s).

### Example 3

### Preparation of N-(3,4-dichlorobenzyl)-N-methyl-5-(4-(trifluoromethylsulfonamido)phenyl)-1,3,4-oxadiazole-1-carboxamide (Compound 17)

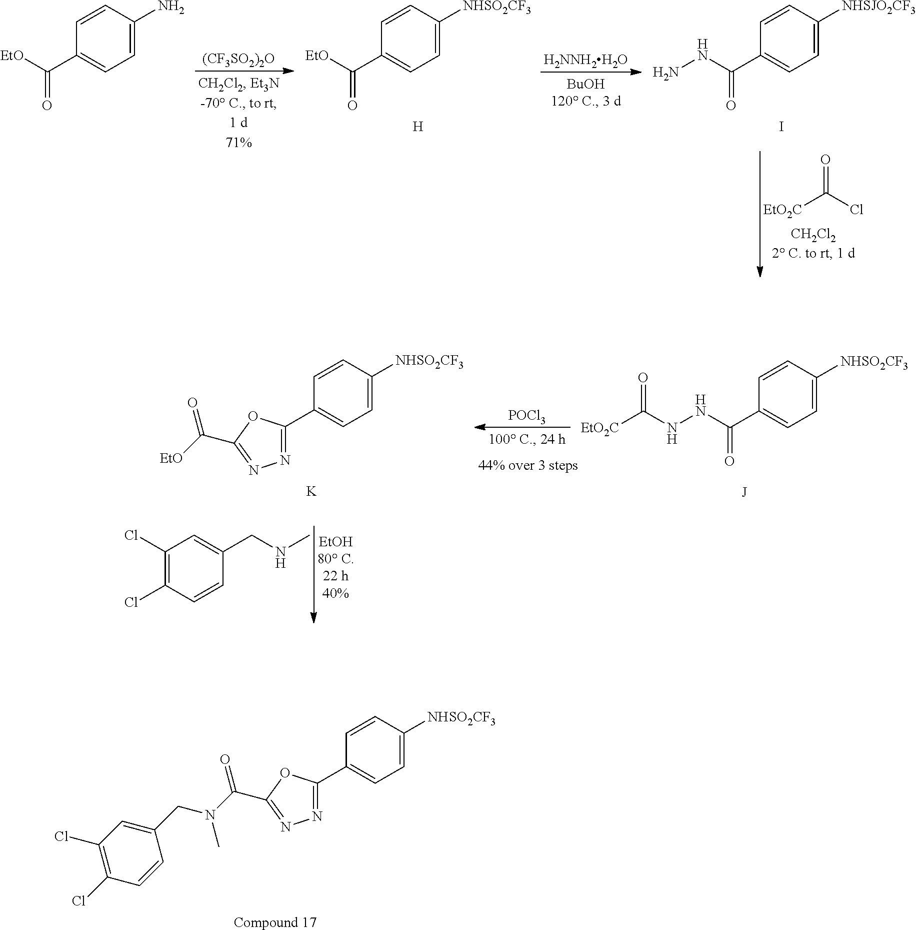

### Step 1: Ethyl 4-(trifluoromethylsulfonamido)benzoate (Compound H)

To a stirred solution of ethyl 4-aminobenzoate (3.30 g, 20 mmol) in anhydrous dichloromethane under nitrogen cooled in an ice-water bath at 3° C. was added, dropwise over 5 min, trifluoromethanesulfonic anhydride (4.1 mL, 24.1 mmol). After 80 min the cooling bath was removed and the mixture was stirred under nitrogen for 26 h. The mixture was recooled with ice-water bath and more trifluoromethanesulfonic anhydride (4.06 mL, 24.1 mmol) was added and the mixture was stirred at room temperature for another 16 h. The mixture was then cooled to −70° C. and triethylamine (6.72 mL, 48.2 mmol) was added dropwise over 20 min, keeping the temperature <−55° C. After the addition was complete the resulting solution was stirred at room temperature for 3 d. The reaction mixture was diluted with dichloromethane (50 mL) and washed with 1 N hydrochloric acid (100 mL), then brine (50 mL). The remaining organic layer was dried (MgSO₄) and evaporated. The residue was purified by flash chromatography (silica gel, 15% EtOAc/petroleum ether) to afford 4.24 g (71%) of the title compound as a white solid.

### Step 2: 1,1,1-Trifluoro-N-(4-(hydrazinecarbonyl)phenyl)methanesulfonamide (Compound I)

To a mixture of ethyl 4-(trifluoromethylsulfonamido)benzoate (4.24 g, 14.3 mmol) in 1-butanol (15 mL) was added hydrazine monohydrate (3.30 mL, 71.3 mmol) and the mixture was heated at 120° C. for 3 d. The solvent was evaporated and the oily residue was azeotroped with ethanol to leave 4.95 g of the crude title compound as an oil.

### Step 3: Ethyl 2-oxo-2-(2-(4-(trifluoromethylsulfonamido)benzoyl)hydrazinyl)acetate (Compound J)

To a stirred mixture of crude 1,1,1-trifluoro-N-(4(hydrazinecarbonyl)phenyl)methane sulfonamide (4.95 g) in anhydrous dichloromethane (50 mL) under nitrogen, cooled to 2° C. was added ethyl chlorooxoacetate (3.19 mL, 28.6 mmol) dropwise over 5 min. After 10 min, the cooling bath was removed and stirring was continued for 20 h. The mixture was filtered and the solid was washed with dichloromethane twice and dried at 60° C. under vacuum to give 6.90 g of the crude title compound as a white solid.

### Step 4: Ethyl 5-(4-(trifluoromethylsulfonamido)phenyl)-1,3,4-oxadiazole-2-carboxylate (Compound K)

A mixture of crude ethyl 2-oxo-2-(2-(3-(trifluoromethylsulfonamido)benzoyl)hydrazinyl)acetate (6.90 g) in phosphorous oxychloride (200 mL) was stirred at 100° C. for 24 h. The excess POCl₃ was evaporated and the residue was allowed to stand for 6 d. At this time the residue was partitioned between water (200 mL) and dichloromethane (400 mL), using a little methanol (5 mL) to dissolve the remaining solid in the flask to transfer to a separating funnel. The layers were separated and aqueous layer was extracted further with dichloromethane (2×300 mL) and the combined organic extracts were washed with brine (50 mL), dried (MgSO₄) and evaporated. The residue was preabsorbed onto silica gel and purified by flash chromatography (silica gel, 2% MeOH/CH₂Cl₂) to give 2.31 g (44% over 3 steps) of the title compound as a pale pink solid.

### Step 5: N-(3,4-Dichlorobenzyl)-N-methyl-5-(4-(trifluoromethylsulfonamido)phenyl)-1,3,4-oxadiazole-2-carboxamide (Compound 17)

A mixture of ethyl 5-(4-(trifluoromethylsulfonamido)phenyl)-1,3,4-oxadiazole-2-carboxylate (0.9 mL g, 0.083 mmol) and N-(3,4-dichlorobenzyl)-N-methylamine (47.4 mg, 0.250 mmol) in ethanol (5 mL) was stirred at 80° C. under nitrogen for 22 h. After this time the reaction mixture was concentrated in vacuo. The resulting residue was purified by preparative HPLC. This gave 17.1 mg (40%) of the title compound as a white solid. ¹H NMR δ (ppm) (DMSO-d₆): 2.99 (3H, s), 3.60-4.06 (1H, br s), 4.75 and 5.04 (2H, s), 7.35-7.43 (3H, m), 7.63-7.70 (2H, m), 7.94 (1H, d, J=8.43 Hz), 8.00 (1H, d, J=8.30 Hz). Rotameric effect observed in NMR; LCMS (10 cm_ESI_Bicarb) t_{R} 3.01 min; m/z 017/509/511 [M−H]⁻

Following the procedure set forth above, but employing a suitable amine in Step 5, following compounds were prepared:

### N-(3,4-dichlorobenzyl)-N-methyl-5-(4-(trifluoromethylsulfonamido)phenyl)-1,3,4-oxadiazole-2-carboxamide (Compound 17)

LCMS (10 cm_ESI_Bicarb) Rt 3.01 min; m/z. 507/509/511 [M−H]−; ¹H NMR δ (ppm) (DMSO-d₆): 2.99 (3H, s), 3.60-4.06 (1H, br s), 4.75 and 5.04 (2H, s), 7.35-7.43 (3H, m), 7.63-7.70 (2H, m), 7.94 (1H, d, J=8.43 Hz), 8.00 (1H, d, J=8.30 Hz). Rotameric effect observed in NMR.

### N-(4-tert-butylbenzyl)-5-(4-(trifluoromethylsulfonamido)phenyl)-1,3,4-oxadiazole-2-carboxamide (Compound 20)

LCMS (10 cm_ESI_Bicarb) Rt 3.06 min; m/z 483/484/485 [M+H]−; ¹H NMR δ (ppm) (DMSO-d₆): 1.25 (9H, s), 4.43 (2H, s). 7.27 (2H, d, J=7.99 Hz), 7.34 (4H, s), 7.96 (2H, d, J=8.09 Hz), 9.80 (1H, s). Sulfonamide NH not observed.

### N-(4-phenoxybenzyl)-5-(4-(trifluoromethylsulfonamido)phenyl)-1,3,4-oxadiazole-2-carboxamide (Compound 23)

LCMS (10 cm_ESI_Bicarb) Rt 2.99 min; m/z 517/518/519 [M−H]−; ¹H NMR δ (ppm) (DMSO-d₆): 4.46 (2H, d, J=6.19 Hz), 6.95-6.99 (4H, m), 7.11 (1H, t, J=7.32 Hz), 7.34-7.40 (6H, m), 8.00 (2H, d, J=8.34 Hz), 9.85 (1H, t, J=6.12 Hz). Sulfonamide NH not observed.

### 1,1,1-trifluoro-N-(4-(5-(4-(3-(trifluoromethyl)phenyl)piperazine-1-carbonyl)-1,3,4-oxadiazol-2-yl)phenyl)methanesulfonamide (Compound 25)

LCMS (10 cm_ESI_Bicarb) Rt 3.01 min; m/z 550/551/552 [M+H]−; ¹H NMR δ (ppm) (DMSO-d₆): 3.39 (4H, t, J=4.91 Hz), 3.84 (2H, t. J=5.05 Hz), 4.14 (2H, t, J=4.85 Hz), 7.10 (1H, d, J=7.61 Hz), 7.21-7.29 (2H, m), 7.39-7.48 (3H, m), 8.02 (2H, d. J=8.32 Hz). Sulfonamide NH not observed.

### Formulation Examples

### Formulation Preparation 1

Hard gelatin capsules containing the following ingredients are prepared:

| Ingredients | Quantity (mg/capsule) |
|---|---|
| active ingredient | 30.0 |
| starch | 305.0 |
| magnesium stearate | 5.0 |

The above ingredients are mixed and tilled into hard gelatin capsules in 340 mg quantities.

### Formulation Preparation 2

A tablet formula is prepared using the ingredients below:

| | Quantity |
|---|---|
| Ingredients | (mg/tablet) |
| active ingredient | 25.0 |
| cellulose, | 200.0 |
| microcrystalline | |
| colloidal silicon dioxide | 10.0 |
| stearic acid | 5.0 |

The components are blended and compressed to form tablets, each weighing 240 mg.

### Example 1

### Effect on Calcium Activated Chloride Channel

### Material and Conditions

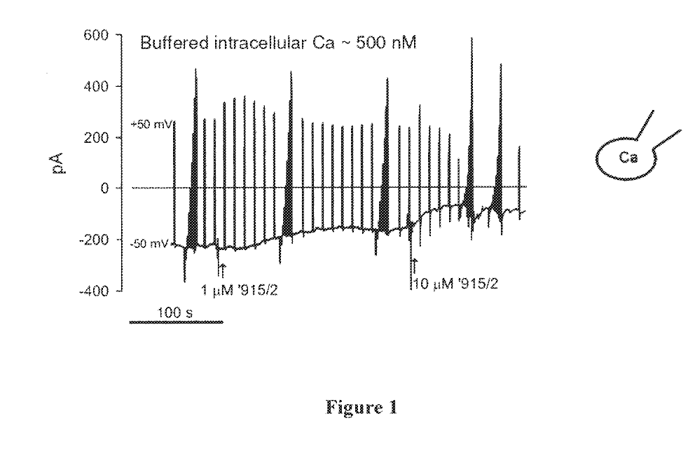

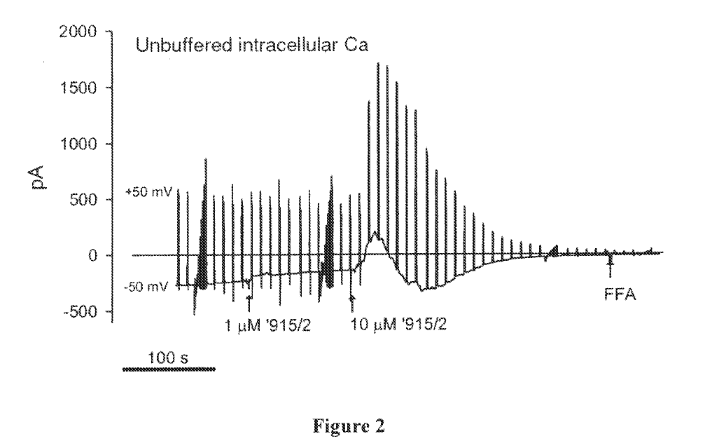

Effect on calcium-activated chloride channels (CaCC) was investigated in JME/CF15 cells (see Jefferson et al. “Expression of normal and cystic fibrosis phenotypes by continuous airway epithelial cell lines” Am J. Physiol. (1990) December; 259 (6 Pt 1): L496-505). These cells do not express CFTR, but express CaCC-mediated currents. Test compound was Compound 10 ('915/2 in FIGS. 1 and 2) and positive control for CaCC was flufenamic acid (FFA).

JME/CF15 cells were prepared for whole cell patch clamp recordings following standard protocols (as described in Fischer et al. “Basolateral Cl channels in primary airway epithelial cultures” Am J Physiol Lung Cell Mol Physiol. (2007) 292:L1432-L1443, which is incorporated herein by reference in its entirety) while incubated in a bath solution as follows (mM): 140 HCl, 158 N-methyl-D-glucamine (NMDG), 2 CaCl₂, 1 MgCl₂, 12.5 Hepes, 10 glucose, pH 7.4. To selectively activate calcium-activated chloride channels (CaCC), the pipette solution consisted of (in mM) HCl, 160 N-methyl-D-glucamine (NMDG), 1 MgCl₂, 5 Hepes, 1 glucose, 5 MgATP, pH 7.4.

Conventional whole cell patch clamp recordings were made. Effects of compounds were tested in 2 runs at 1 and 10 μM, respectively, on CaCC. To activate CaCC mediated Cl⁻ conductances, the pipette solution contained 500 nM CaCl₂ without EGTA (in order to allow for agonist induced regulation of CaCC) or a mixture of 1.8 mM CaCl₂ and 2 mM EGTA resulting in a free concentration of calcium of 500 nM to maintain a stable intracellular calcium concentration.

CaCC-mediated Cl⁻ currents were measured with a pipette solution that contained ˜500 nM free Ca to activate CaCC. Under conditions where intracellular calcium was stably buffered (with EGTA) addition of compound 10 to the bath, blocked currents (FIG. 1). Under conditions where intracellular calcium was unbuffered (low EGTA) addition of 10 μM of compound 10 showed an additional effect (FIG. 2): it transiently activated an additional conductance (note the large increase in pulse height). This is likely a Ca-activated K⁺ conductance because i) it generates a large outward current (which rules out a Cl⁻ conductance), and ii) it is transient (likely because intracellular K⁺ is exhausted). The remaining chloride current is eventually well blocked by compound 10, and 100 μM FFA blocks only slightly more.

The experimental conditions were not set up to measure a K⁺ conductance, i.e., there is no K⁺ in the employed solutions, and the measured currents are dependent on the residual K⁺ found in cells. Nevertheless, the activation of a K⁺ conductance by compound 10 in unbuffered (hut not in Ca-buffered) solutions indicated an activation of a Ca-activated K⁺ conductance by this compound. In an epithelial setting, concurrent block of CaCC and a K⁺ conductance may result in weaker blocker efficiency or even in stimulation of secretion.

### Example 2

### In Vivo Study

For in vivo studies for the treatment of diarrhea, mice (CD1 strain, approximately 25 g) were deprived of food for at least 20 hours and anaesthetized with an intraperitoneal injection of ketamine (80 mg/kg) and xylazine (16 mg/kg) prior to surgery. Anesthesia was maintained as needed. Body temperature was maintained using a heated operating table. The abdominal area was shaved and disinfected with 70% alcohol swabs. An incision was made on the abdomen for exposure of the small intestine. Following the abdominal incision two different closely-spaced locations of the small intestine were isolated and looping was performed. Loop 1 started around 6 cm from the junction of stomach and duodenum. Loop 1 and Loop 2 were intestinal loops of around 25 mm in length with inter-loop space of around 5-10 mm. One hundred microliters of the PBS pH 8.5 or the PBS pH 8.5 containing 2.0 μg cholera toxin (CTX) (with or without compound 10) was injected into each loop. The abdominal incision was then closed with sutures and mice were allowed to recover from anesthesia. During this recovery period, close monitoring was performed. At 4 hours after the injection of the compound 1.0 or control compound dose formulation, the mice were euthanized via CO₂ inhalation plus diaphragm severance, the intestinal loops were exteriorized, and loop length and loop weight were measured after removal of mesentery and connective tissue to quantify the net fluid secretion (measured as g/cm of loop).

For the dose formulation observation, compound 10 at 1.0 mg/mL with or without CTX looked like colorless solution. The positive control BF'032 (3-(3,5-dibromo-4-hydroxyphenyl)-N-(4-phenoxybenzyl)-1,2,4-oxadiazole-5-carboxamide) with CTX looked like yellowish solution right after preparation, and changed to milk-white emulsion about 1.5 hours after preparation, and BF'032 without CTX looked liked light yellowish solution.

Based on the data, compound 10 at 10 μg/loop, 100 μg/loop, and BF'032 (positive control) at 100 μg/loop showed statistically significant inhibition.

TABLE 3

| | | | | | % |
|---|---|---|---|---|---|
| | | Mean | Std Dev | SEM | Inhibition |
| Vehicle | CTX | 0.166188 | 0.073003 | 0.023102 | |
| | PBS | 0.041397 | 0.008365 | 0.002647 | |
| 10 μg | CTX | 0.076453 | 0.047872 | 0.015149 | 71.77% |
| compound 10 | | | | | |
| | PBS | 0.040145 | 0.004458 | 0.001411 | |
| 100 μg | CTX | 0.049957 | 0.017774 | 0.005625 | 92.14%^{§} |
| compound 10 | | | | | |
| | PBS | 0.040145 | 0.004458 | 0.001411 | |
| 100 μg | CTX | 0.092453 | 0.048095 | 0.01522 | 62.69%^{ψ} |
| BF000710032 | | | | | |
| | PBS | 0.045897 | 0.01196 | 0.003785 | |

^{§}P < 0.001, ***
^{ψ}P < 0.05, *

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method for blocking a transport of a halide ion across a calcium activated chloride channel (CaCC), comprising contacting the CaCC with an effective amount of a compound of formula I: 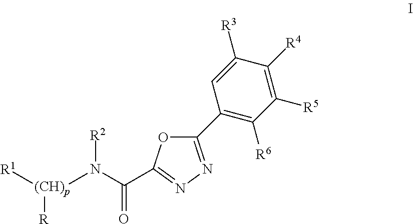
wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
or R¹ and R² are taken together with the nitrogen atom to which they are bonded to form a heterocycle or substituted heterocycle;
R³, R⁴, and R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;
or a pharmaceutically acceptable salt, isomer, or tautomer thereof,
thereby blocking the transport of the halide ion across the CaCC.

2. The method of claim 1, wherein the contacting is in vitro.

3. The method of claim 1, wherein the contacting is in vivo.

4. A method of treating a disease in an animal, which disease is responsive to blocking of a calcium activated chloride channel (CaCC) in the animal, comprising administering to an animal in need thereof an effective amount of a compound of formula I: 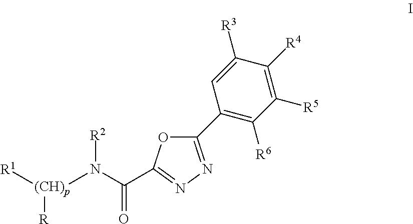
wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
or R¹ and R² are taken together with the nitrogen atom to which they are bonded to form a heterocycle or substituted heterocycle;
R³, R⁴, and R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;
or a pharmaceutically acceptable salt, isomer, or tautomer thereof,
thereby treating the disease in the animal.

5. An in vitro method for blocking a transport of a halide ion across a calcium activated chloride channel (CaCC), comprising contacting the CaCC with an effective amount of a compound of formula I: 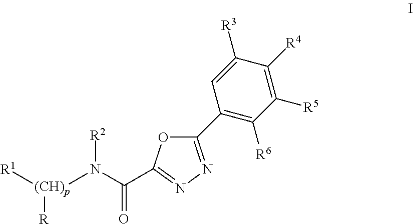
wherein
p is 0, 1, 2, or 3;
R is independently selected from the group consisting of hydrogen and alkyl;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
or R¹ and R² are taken together with the nitrogen atom to which they are bonded to form a heterocycle or substituted heterocycle;
R³, R⁴, and R⁵ are each independently selected from the group consisting of hydrogen, halo, hydroxyl, aminocarbonyl, and sulfonylamino; and
R⁶ is selected from the group consisting of hydrogen, hydroxyl, alkoxy and substituted alkoxy;
or a pharmaceutically acceptable salt, isomer, or tautomer thereof,
thereby blocking the transport of the halide ion across the CaCC.

6. The method of claim 1, wherein the compound inhibits halide ion transport by CaCC.

7. The method of claim 4, wherein the disease is selected from the group consisting of chronic obstructive pulmonary disease (COPD), an inflammatory lung disease, stroke, and an acute or chronic infectious disease.

8. The method of claim 4, wherein the disease is selected from the group consisting of asthma, bronchitis, cystic fibrosis, emphysema, gastrointestinal malabsorption syndrome, steatorrhea, secretory diarrhea, inflammatory diarrhea, allergic inflammation, airway inflammation, inflammatory bowel disease, infectious diarrhea, polycystic kidney disease (PKD), cardiac arrhythmia, male infertility and disorders associated with neovascularization.

9. The method of claim 4, wherein the disease is selected from the group consisting of olfactory and taste disorders; ophthalmic angiogenesis related disease; neuronal disorders; cardiovascular disease; obstructive or inflammatory airway disease; diarrhea and/or urinary incontinence; kidney disease; bone metabolic disease; diseases that are responsive to inhibition of angiogenesis; and diseases that is responsive to reduction of intraocular pressure.

10. The method of claim 4, wherein the disease is a cardiovascular disease selected from the group consisting of atherosclerosis, ischemia, reperfusion injury, hypertension, restenosis, arterial inflammation, and ischaemic heart disease.

11. The method of claim 4, wherein the compound is administered by a parenteral or transdermal route.

12. The method of claim 11, wherein the parenteral route is selected from the group consisting of intravenous, intramuscular, intraperitoneal and subcutaneous administration.

13. The method of claim 4, wherein the compound is administered by an oral route or by inhalation.

14. The method of claim 4, wherein the compound is formulated for oral administration in a formulation selected from the group consisting of capsules, tablets, elixirs, suspensions and syrups.

15. The method of claim 4, wherein the compound is formulated as a controlled release formulation.

16. The method of claim 4, wherein the compound is administered in combination with a second agent for the treatment of the disease.

17. The method of claim 16, wherein the second agent is selected from the group consisting of expectorants, mucolytics, antibiotics, anti-histamines, steroids, anti-inflammatory agents, and decongestants.

18. The method of claim 1, wherein R is hydrogen or methyl.

19. The method of claim 1, wherein R⁶ is hydrogen.

20. The method of claim 1, wherein each of R³ and R⁵ is independently halo and R⁴ is hydrogen or hydroxyl.

21. The method of claim 20, wherein R⁴ is hydroxyl.

22. The method of claim 1, wherein p is 0 or 1.

23. The method of claim 1, wherein R² is hydrogen or methyl.

24. The method of claim 1, wherein each of R³, R⁵, and R⁶ is hydrogen; and R⁴ is sulfonylamino.

25. The method of claim 1, wherein each of R³, R⁴, and R⁶ is hydrogen; and R⁵ is sulfonylamino.

26. The method of claim 1, wherein R¹ is alkyl, substituted alkyl, aryl, or substituted aryl.

27. The method of claim 1, wherein the compound is represented by formula II: 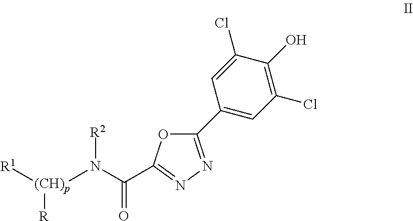
wherein R, R¹, R², and p are as defined in claim 1.

28. The method of claim 27, wherein p is 0 or 1.

29. The method of claim 28, wherein R is hydrogen or methyl.

30. The method of claim 28, wherein R² is hydrogen or methyl.

31. The method of claim 30, wherein p is 1 and R¹ is substituted alkyl or substituted aryl.

32. The method of claim 27, wherein p is 0 or 1; R is hydrogen or methyl; R¹ is substituted alkyl substituted with aryl or substituted aryl substituted with halo, alkyl, substituted alkyl, aryloxy, substituted alkoxy, or aryl; and R² is hydrogen or methyl.

33. The method of claim 1, wherein the compound is represented by formula III: 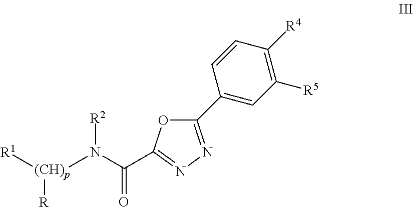
wherein
R, R¹, R², and p are as defined in claim 1, and
R⁴ and R⁵ are each independently selected from the group consisting of hydrogen and sulfonylamino.

34. The method of claim 33, wherein R is hydrogen or methyl.

35. The method of claim 34, wherein p is 0 or 1.

36. The method of claim 35, wherein R² is hydrogen or methyl.

37. The method of any of claim 36, wherein p is 1 and R¹ is aryl or substituted aryl.

38. The method of claim 33, wherein p is 0 or 1; R is hydrogen or methyl; R¹ is aryl or substituted aryl; R² is hydrogen or methyl; R⁴ is hydrogen; and R⁵ is sulfonylamino.

39. The method of claim 33, wherein p is 0 or 1; R is hydrogen or methyl; R¹ is aryl or substituted aryl; R² is hydrogen or methyl; R⁵ is hydrogen; and R⁴ is sulfonylamino.

40. The method of claim 1, wherein the compound is represented by formula IV: 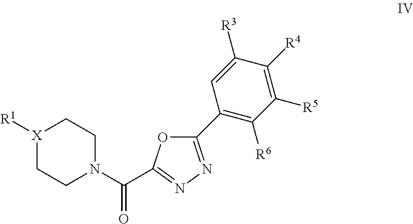
wherein
X is CH or N; and
R¹, R³, R⁴, R⁵, and R⁶ are as defined in claim 1.

41. The method of claim 40, wherein X is CH.

42. The method of claim 41, wherein X is N.

43. The method of claim 42, wherein R⁶ is hydrogen.

44. The method of claim 43, wherein each of R³ and R⁵ is independently halo; and R⁴ is hydroxyl.

45. The method of claim 40, wherein each of R³, R⁴, and R⁶ is hydrogen; and R⁵ is sulfonylamino.

46. The method of claim 40, wherein each of R³, R⁵, and R⁶ is hydrogen; and R⁴ is sulfonylamino.

47. The method of claim 40, wherein R¹ is alkyl, substituted alkyl, aryl, or substituted aryl.

48. The method of claim 1, wherein the compound is:
5-(3,5-dichloro-4-hydroxyphenyl)-N-(3-(trifluoromethoxy)benzyl)-1,3,4-oxadiazole-2-carboxamide;
5-(3,5-dichloro-4-hydroxyphenyl)-N-(4-phenoxybenzyl)-1,3,4-oxadiazole-2-carboxamide;
(4-benzylpiperidin-1-yl)(5-(3,5-dichloro-4-hydroxyphenyl)-1,3,4-oxadiazol-2-yl)methanone;
(5-(3,5-dichloro-4-hydroxyphenyl)-1,3,4-oxadiazole-2-yl)(4-(3-trifluoromethyl)phenyl)piperazin-1-yl)methanone;
N-(4-tert-butylbenzyl)-5-(3,5-dichloro-4-hydroxyphenyl)-1,3,4-oxadiazole-2-carboxamide;
N-benzhydryl-5-(3,5-dichloro-4-hydroxyphenyl)-1,3,4-oxadiazole-2-carboxamide;
N-(4-phenoxybenzyl)-5-(3-(trifluoromethylsulfonamido)phenyl)-1,3,4-oxadiazole-2-carboxamide
N-(3-(5-(4-benzylpiperidine-1-carbonyl)-1,3,4-oxadiazol-2-yl)phenyl)-1,1,1-trifluoromethanesulfonamide;
N-(4-tert-butylbenzyl)-5-(3-(trifluoromethylsulfonamido)phenyl)-1,3,4-oxadiazole-2-carboxamide;
N-(3,4-dichlorobenzyl)-N-methyl-5-(trifluoromethylsulfonamido)phenyl)-1,3,4-oxadiazole-2-carboxamide;
1,1,1-trifluoro-N-(3-(5-(4-(3-(trifluoromethyl)phenyl)piperazine-1-carbonyl)-1,3,4-oxadiazol-2-yl)phenyl)methanesulfonamide;
5-(3,5-dichloro-4-hydroxyphenyl)-N-(3,4-dichlorobenzyl)-N-methyl-1,3,4-oxadiazole-2-carboxamide;
5-(3,5-dichloro-4-hydroxyphenyl)-N-methyl-N-(3-phenoxybenzyl)-1,3,4-oxadiazole-2-carboxamide;
5-(3,5-dichloro-4-hydroxyphenyl)-N-(3-phenoxybenzyl)-1,3,4-oxadiazole-2-carboxamide;
5-(3,5-dichloro-4-hydroxyphenyl)-N-(2,2-diphenylethyl)-1,3,4-oxadiazole-2-carboxamide;
N-(3-(benzyloxy)benzyl)-5-(3,5-dichloro-4-hydroxyphenyl)-1,3,4-oxadiazole-2-carboxamide;
N-(3,4-dichlorobenzyl)-N-methyl-5-(4-(trifluoromethylsulfonamido)phenyl)-1,3,4-oxadiazole-2-carboxamide;
N-(4-(benzyloxy)benzyl)-5-(3,5-dichloro-4-hydroxyphenyl)-1,3,4-oxadiazole-2-carboxamide;
N-(biphenyl-3-ylmethyl)-5-(3,5-dichloro-4-hydroxyphenyl)-1,3,4-oxadiazole-2-carboxamide;
N-(4-tert-butylbenzyl)-5-(4-(trifluoromethylsulfonamido)phenyl)-1,3,4-oxadiazole-2-carboxamide;
5-(3,5-dichloro-4-hydroxyphenyl)-N-(3-fluoro-5-trifluoromethyl)benzyl)-1,3,4-oxadiazole-2-carboxamide;
5-(3,5-dichloro-4-hydroxyphenyl)-N-(4-(trifluoromethoxy)-1,3,4-oxadiazole-2-carboxamide;
N-(4-phenoxybenzyl)-5-(4-(trifluoromethylsulfonamido)phenyl)-1,3,4-oxadiazole-2-carboxamide;
5-(3,5-dichloro-4-hydroxyphenyl)-N-(4-fluoro-3-(trifluoromethyl)benzyl)-1,3,4-oxadiazole-2-carboxamide;
1,1,1-trifluoro-N-(4-(5-(4-(3-(trifluoromethyl)phenyl)piperazine-1-carbonyl)-1,3,4-oxadiazol-2-yl)phenyl)methanesulfonamide; or
N-(1-(4-chlorophenyl)ethyl)-5-(3,5-dichloro-4-hydroxyphenyl)-1,3,4-oxadiazole-2-carboxamide, or
a pharmaceutically acceptable salt, isomer, or tautomer thereof.

49. The method of claim 1, wherein the compound is in a composition which further comprises a pharmaceutically acceptable carrier.

50. The method of claim 1, wherein the halide ion is Cl⁻.

51. The method of claim 1, wherein the channel is present in an animal cell selected from the group consisting of epithelial cell, bipolar cell, smooth muscle cell, acinar and duct cell of lachrymal, parotid, submandibular, and/or sublingual gland, endothelial cell, and kidney cell.
